(19) United States
(12) Patent Application Publication
(10) Pub. No.: US 2023/0183320 A1
(43) Pub. Date: Jun. 15, 2023

(54) RECOMBINANT FIBCD1 AND USE THEROF IN THE TREATMENT OF MUSCLE ATROPHY

(71) Applicant: ST. JUDE CHILDREN’S RESEARCH HOSPITAL, INC., Memphis, TN (US)

(72) Inventors: Fabio DEMONTIS, Memphis, TN (US); Liam HUNT, Memphis, TN (US); Flavia GRACA, Memphis, TN (US); Mamta RAI, Memphis, TN (US)

(21) Appl. No.: 17/801,907

(22) PCT Filed: Feb. 24, 2021
(86) PCT No.: PCT/US2021/019338

§ 371 Date: Aug. 24, 2022

Related U.S. Application Data
(60) Provisional application No. 62/983,077, filed on Feb. 28, 2020.

(51) Int. Cl. C07K 14/75; C12N 15/63; A61K 38/36; A61P 21/00

(52) U.S. Cl. CPC C07K 14/75; A61K 38/363; A61P 21/00; C12N 15/63; C07K 2319/43

(57) ABSTRACT

Recombinant Fibrinogen C Domain Containing 1 (rFibcd1) proteins and methods for using the same in the treatment of muscle atrophy are provided as are vectors, host cells, pharmaceutical compositions and modified RNA molecules encoding the rFibcd1 proteins.

### INTRODUCTION

This patent application claims the benefit of priority from U.S. Provisional Serial No. 62/983,077 filed Feb. 28, 2020, the content of which is incorporated herein by reference in its entirety.

### BACKGROUND

Skeletal muscle wasting, defined as the loss of muscle mass and strength, is a complication of many common diseases. These diseases include neuromuscular disorders (e.g., muscular dystrophies, spinal muscular atrophy, juvenile myositis, and other myopathies and motor neuron diseases), cancers (e.g., ~50% of solid tumors), chronic infections (e.g., AIDS and tuberculosis), inherited mitochondrial diseases, and type 1 diabetes. In addition, muscle wasting is a side effect of several commonly used drugs, including corticosteroids, neuromuscular blockers (used as an adjunct to anesthesia), antibiotics (e.g., aminoglycosides and colistin), and chemotherapy. Moreover, hospitalization, best rest, traumas, and improper diet and lifestyle can also contribute to loss of muscle mass in children.

The loss of skeletal muscle mass has a negative effect on quality of life and increases the risk of mortality from many causes, whereas muscle hypertrophy protects from disease progression. For example, promoting muscle growth by inhibiting myostatin, a negative regulator of muscle mass, significantly reduces diabetes symptoms. Preventing muscle mass loss in tumor-bearing mice improves prognosis and prolongs their survival even if cancer growth and progression are not halted, indicating that muscle wasting improves disease outcome and patient survival. Thus, preserving skeletal muscle mass can improve physiologic homeostasis and patient survival in the context of many human diseases.

Muscle mass is determined by the number and size of myofibers, the syncytial muscle cells that compose the muscle. The number of myofibers is mostly determined during development and results from the fusion of myoblasts, the muscle precursor cells. Once formed, myofibers are remodeled in response to physiologic and pathologic challenges. For example, prolonged starvation or malnutrition leads to a decrease in myofiber size (atrophy) due to muscle protein degradation and the release of gluconeogenic amino acids that are used as energy substrates in the liver. In addition to proper diet, neuronal stimulation and muscle contraction are necessary to maintain myofiber size and muscle mass. Reduced muscle contractile capacity (seen in muscular dystrophies and other myopathies) or insufficient neuronal stimulation of muscle (observed in neuromuscular disorders) leads to myofiber atrophy and muscle wasting. Lastly, cancer cells secrete cytokines that induce muscle wasting (cachexia) and release of gluconeogenic substrates, which fuel cancer growth. Thus, muscle wasting due to myofiber atrophy is induced in many disease contexts.

In the past decades, there has been growing appreciation that skeletal muscle secretes hundreds of signaling factors, known as myokines (Deshmukh, et al. (2015) J. Proteome Res. 14:4885-4895). Interestingly, some myokines regulate myofiber size in an autocrine/paracrine manner (Pedersen & Febbraio (2012) Nature Reviews. Endocrinology 8:457-465; Hunt, et al. (2015) Genes Dev. 29:2475-2489; Rai & Demontis (2016) Annu. Rev. Physiol. 78:85-107), as exemplified by postnatal knockout of the myokine myostatin, which increases myofiber size and muscle mass (Argiles, et al. (2012) Drug Discov Today 17:702-709; Lee (2004) Annu. Rev. Cell Dev. Biol. 20:61-86; Whittemore, et al. (2003) Biochem. Biophys. Res. Commun. 300:965-971). However, apart from extensive efforts in developing myostatin inhibitors (Murphy, et al. (2010) Am. J. Physiol. Regul. Integr. Comp. Physiol. 301:R716-726; Smith & Lin (2013) Curr. Opin. Support. Palliat. Care 7:352-36) and few other notable studies (Suriben, et al. (2020) Nat. Med. 26:1264-1270; Vinel, et al. (2018) Nat. Med. 24:1360-1371; Benoit, et al. (2017) Nat. Med. 23:990-996).

Studies have revealed much about the cellular and molecular mechanisms of skeletal muscle atrophy in different disease contexts. These studies have deciphered the signaling pathways responsible for skeletal muscle wasting, thereby highlighting several potential pharmacologic targets for therapies for muscle wasting. However, despite this increased understanding of the mechanisms involved, it has been difficult to develop pharmacologic interventions for muscle wasting. In fact, some of the key regulators of muscle atrophy are not easily druggable (e.g., transcription factors) or have such a general role in tissue homeostasis (e.g., Akt and mTOR) that their pharmacologic modulation is likely to induce adverse effects, including cancer. Thus, new approaches for treating skeletal muscle wasting are needed.

### SUMMARY OF THE INVENTION

This invention provides a recombinant Fibrinogen C Domain Containing 1 (Fibcd1) protein fragment of, or variant or derivative thereof, wherein said fragment includes the fibrinogen-related domain. In some embodiments, the fragment is less than 400 amino acid residues in length. In other embodiments, the fragment includes residues 241-457 of SEQ ID NO:2, or an ortholog thereof. A vector, host cell and modified RNA molecule harboring a nucleic sequence encoding the recombinant fragment is also provided as is pharmaceutical composition including the recombinant fragment and a pharmaceutically acceptable carrier.

This invention further includes a fusion protein composed of a Fibcd1 protein, e.g., a Fibcd1 fragment, variant or derivative, and a second polypeptide, e.g., an epitope or cell-penetrating peptide, as well as a pharmaceutical composition including said fusion protein in admixture with a pharmaceutically acceptable carrier.

This invention also provides methods of treating muscle atrophy in a subject by administering to a subject in need of treatment an effective amount of the recombinant fragment, vector, fusion protein or modified RNA. In some embodiments, the muscle atrophy is associated with aging, injury, disuse, cachexia, nutritional or metabolic derangements, vascular insufficiency, drug treatment or a neuromuscular disorder or disease.

### BRIEF DESCRIPTION OF THE DRAWINGS

FIG. 1 shows that siRNAs for mouse Fibcd1 induce mouse C2C12 myotube atrophy. NT or Fibcd1 siRNAs were transfected into mouse C2C12 myotube-enriched cultures for 48 hours, followed by treatment for further 24 hours with rFibcd1 or a vehicle control. Representative images of myotubes were stained for myosin heavy chain. Measurement of myotube width indicates that Fibcd1 siRNAs induce atrophy and that myotube size is rescued by rFibcd1. Data are means±SD with n≥50 myotubes/group. ****P<0.0001 and ^{&&}P<0.01, compared to the indicated control; P values were determined by two-way ANOVA with Sidak’s multiple comparisons test.

FIGS. 2A-2B show that rFibcd1 rescues LLC cancer-induced myofiber atrophy. Shown is the quantification of myofiber size from diaphragm muscles of control mice (n=10), control mice treated with rFibcd1 (n=10), mice that carry LLC tumors (n=8), and mice that carry LLC tumors and that are treated with rFibcd1 (n=9). Treatment with rFibcd1 included 3 intraperitoneal injections of rFibcd1 at 1 mg/Kg or mock injection of the vehicle (1% BSA). FIG. 2A, Feret’s minimal diameter of type 1, 2a, and 2x/2b myofibers. FIG. 2B, Feret’s minimal diameter for each myofiber type normalized to the corresponding myofiber size of control mice. Treatment with rFibcd1 partially rescues cancer-induced myofiber atrophy.

FIG. 3 shows that rFibcd1 rescues myofiber atrophy induced by a patient-derived melanoma xenograft. Shown is quantification of myofiber size from diaphragm muscles of control mice with a non-cachectic melanoma (n=10), control mice with a non-cachectic melanoma and that are treated with rFibcd1 (n=10), mice that carry a cachectic melanoma (n=10), and mice that carry a cachectic melanoma and that are treated with rFibcd1 (n=10). Treatment with rFibcd1 included 3 intraperitoneal injections of rFibcd1 at 3 mg/Kg or mock injection of the vehicle (1% BSA). Shown are Feret’s minimal diameters of type 1, 2a, and 2x/2b myofibers.

FIG. 4 shows that rFibcd1 rescues dexamethasone-induced myofiber atrophy.

### DETAILED DESCRIPTION OF THE INVENTION

It has now been found that a recombinant Fibcd1 variant (rFibcd1, i.e., recombinant Fibrinogen C Domain Containing 1) rescues the decrease in muscle cell size (myofiber atrophy), which is a feature of many human disease conditions including, e.g., cancer, aging, diabetes, neurological disorders, and infections, and a worsening factor for prognosis. In particular, it has been found that muscle cell atrophy induced by FIBCD1 siRNA or dexamethasone treatment can be rescued by rFibcd1. In addition, injection of rFibcd1 injection into mouse muscles can partially rescue cancer-induced reduction in muscle cell size. Accordingly, the invention provides a recombinant Fibcd1 protein (rFibcd1) and a nucleic acid molecule encoding the same (i.e., gene therapy) for promoting Fibcd1 expression in human skeletal muscle, so that disease-associated myofiber atrophy is prevented or ameliorated.

As described in the art, Fibcd1 is a conserved type II transmembrane endocytic receptor, which shows a high-affinity and calcium-dependent binding to acetylated structures such as chitin, some N-acetylated carbohydrates, and amino acids, but not to their non-acetylated counterparts. The term “fibrinogen C domain containing 1” or “Fibcd1” includes isoforms and orthologs of human Fibcd1, which are naturally expressed by cells or are expressed on cells transfected with the Fibcd1 gene. A synonym (alias) of FIBCD1, as recognized in the art, is FLJ14810. The human FIBCD1 gene has external IDs: 25922 (HGNC); 84929 (Entrez Gene); and ENSG00000130720 (Ensembl). The sequence of the wild-type human Fibcd1 protein is known and available under UniProt Accession No. Q8N539 and NCBI Reference Sequence NP_116232.

The cDNA sequence encoding for the wild-type human Fibcd1 receptor is shown in SEQ ID NO:1 and may also include orthologs that encode Fibcd1 proteins that share at least 90% identity with SEQ ID NO:2, e.g., at least 92% identical, at least 95% identical, at least 96% identical, at least 97% identical, at least 98% identical, or at least 99% identical.

Human Fibcd1 protein as defined herein by SEQ ID NO:2, may also include orthologs that share at least 90% identity with SEQ ID NO:1, e.g., at least 92% identical, at least 95% identical, at least 96% identical, at least 97% identical, at least 98% identical, or at least 99% identical. Examples of orthologs of human Fibcd1 include, but are not limited to, Pan troglodytes Fibcd1 (GENBANK Accession No. XP_016801885; 99.57% identity with human Fibcd1), Gorilla Fibcd1 (GENBANK Accession No. XP_030870778; 99.57% identity with human Fibcd1), Pongo abelii Fibcd1 (GENBANK Accession No. XP_024108211; 97.83% identity with human Fibcd1), Macaca mulatta Fibcd1 (GENBANK Accession No. XP_014972035; 96.53% identity with human Fibcd1), Sus scrofa Fibcd1 (GENBANK Accession No. XP_003122287; 93.06% identity with human Fibcd1), Ovis aries Fibcd1 (GENBANK Accession No. XP_027824120; 91.97% identity with human Fibcd1), Bos taurus Fibcd1 (GENBANK Accession No. XP_010808818; 92.19% identity with human Fibcd1), Rattus norvegicus Fibcd1 (GENBANK Accession No. NP_001101299; 90.67% identity with human Fibcd1), Mus musculus Fibcd1 (GENBANK Accession No. NP_849218; 90.24% identity with human Fibcd1), and Equus caballus Fibcd1 (GENBANK Accession No. XP_023484924; 90.67% identity with human Fibcd1).

As used herein, the terms “protein” and “polypeptide” are used interchangeably to refer to a polymer of linearly arranged amino acid residues linked by peptide bonds, whether produced biologically, recombinantly, or synthetically and whether composed of naturally occurring or non-naturally occurring amino acids. The terms also include proteins that have co-translational (e.g., signal peptide cleavage) and post-translational modifications of the proteins, such as, for example, disulfide-bond formation, glycosylation, acetylation, phosphorylation, proteolytic cleavage, and the like. Furthermore, as used herein, a “protein” refers to a polypeptide that includes modifications, such as deletions, additions, and substitutions (generally conservative in nature, as would be known to a person skilled in the art) to the native sequence, as long as the polypeptide maintains the desired functional activity. These modifications can be deliberate, as through site-directed mutagenesis, or can be accidental, such as through mutations of hosts that produce the proteins, or errors due to PCR amplification or other recombinant DNA methods.

When referred to herein as “recombinant Fibcd1” or “rFibcd1” protein, said protein is produced by recombinant means. “Recombinant Fibcd1” or “rFibcd1” also includes a protein that has co-translational (e.g., signal peptide cleavage) and/or post-translational modifications of the protein, such as, for example, disulfide-bond formation, glycosylation, acetylation, phosphorylation, proteolytic cleavage, and the like.

In addition to wild-type and full-length Fibcd1, the invention also includes a functional fragment, functional variant, or functional derivative of Fibcd1. As used in the context of this invention, a “fragment of Fibcd1,” “variant of Fibcd1,” or “derivative of Fibcd1” is intended to be functional in that said fragment, variant or derivative of Fibcd1 retains the ability to treat or ameliorate one or more symptoms of myofiber atrophy such as, for example, reduction in muscle cell size/mass and/or muscle strength. Accordingly, the term “functional” when used in conjunction with a “fragment,” “derivative” or “variant” refers to a protein molecule that possesses a biological activity that is substantially similar to a biological activity of the wild-type and full-length Fibcd1 from which the fragment, derivative or variant was obtained. By “substantially similar” in this context is meant that the biological activity, e.g., ability to increase in muscle cell size/mass and/or decrease muscle atrophy, is at least 50% as active as a reference, e.g., a corresponding wild-type and full-length Fibcd1, and preferably at least 60% as active, 70% as active, 80% as active, 90% as active, 95% as active, 100% as active or even higher (i.e., the fragment, variant or derivative has greater activity than the wild-type), e.g., 110% as active, 120% as active, or more. Assays to measure the biological activity of a Fibcd1 protein are known in the art, and non-limiting examples are provided herein in the Examples.

In certain embodiments, the Ficbd1 protein is a functional fragment of Fibcd1. A “fragment of Fibcd1” refers to an Fibcd1 protein having an amino-terminal deletion and/or a carboxyl-terminal deletion when compared to the full-length protein. In this respect, a fragment of Fibcd1 has a length that is less than that of the full-length Fibcd1 protein, e.g., a fragment of a human Fibcd1 protein is less than 461 amino acid residues in length. Such fragments may also contain modified amino acids as compared to the full-length protein. In certain embodiments, a fragment of Fibcd1 is about 220 to about 450 amino acids in length. For example, the fragment may be at least 200, 210, 220, 230, 240, 250, 300, 350, 400, or 450 amino acids in length. Useful fragments include Fibcd1 proteins lacking the transmembrane domain located in the N-terminal portion of the protein. In human Fibcd1, the transmembrane domain is located at residues 34-54 of SEQ ID NO:2. Accordingly, a fragment of human Fibcd1 lacks all or a portion of the transmembrane domain, e.g., residues 1-50, 2-50, 1-60, 5-60, 10-70, 20-80, 30-170, or 2-180 of SEQ ID NO:2. In this respect, a fragment of human Fibcd1 includes the 220, 230, 240, 250, 260, 270, 280, 290, 300, 310, 320, 330, 340, 350, 360, 370, 380, 390, or 400 C-terminal amino acid residues of SEQ ID NO:2. In certain aspects, a fragment of Fibcd1 includes at least the fibrinogen-related domain (FReD). In human Fibcd1, the fibrinogen-related domain is located at residues 241-457 of SEQ ID NO: 2. In particular, the fibrinogen-related domain of human Fibcd1 has the amino acid sequence:

PRDCLDVLLSGQQDDGVYSVFPTHYPAGFQVYCDMRTDGGGWTVFQRRED
GSVNFFRGWDAYRDGFGRLTGEHWLGLKRIHALTTQAAYELHVDLEDFEN
GTAYARYGSFGVGLFSVDPEEDGYPLTVADYSGTAGDSLLKHSGMRFTTK
DRDSDHSENNCAAFYRGAWWYRNCHTSNLNGQYLRGAHASYADGVEWSSW
TGWQYSLKFSEMKIRPV (SEQ ID NO:3).

Accordingly, a fragment of human Fibcd1 includes at least residues 175-461, 179-461, 200-461, 240-416, 179-457, 200-457, 241-457 of SEQ ID NO:2. In this respect, a fragment of human Fibcd1 lacks at least the 50, 75, 100, 175, 200, 210, 220, 230, 240 N-terminal residues and/or 1, 2, 3 or 4 C-terminal amino acid residues of SEQ ID NO:2.

A “functional variant of Fibcd1” or “variant of Fibcd1” refers to a protein differing from the naturally occurring protein, i.e., Fibcd1, or nucleic acid encoding such protein, by one or more amino acid or nucleic acid deletions, additions, substitutions or side-chain modifications, yet retains one or more functions or biological activities (i.e., functions or activities specific to muscle) of the naturally occurring molecule, i.e., Fibcd1. Amino acid substitutions include alterations in which an amino acid is replaced with a different naturally-occurring or a non-conventional amino acid residue. Such substitutions may be classified as “conservative,” in which case an amino acid residue contained in a protein is replaced with another naturally occurring amino acid of similar character either in relation to polarity, side chain functionality or size. Substitutions encompassed by variants as described herein may also be “non conservative,” in which an amino acid residue which is present in a protein is substituted with an amino acid having different properties (e.g., substituting a charged or hydrophobic amino acid with alanine), or alternatively, in which a naturally-occurring amino acid is substituted with a non-conventional amino acid. Also encompassed within the term “variant,” when used with reference to a nucleic acid molecule or protein, are variations in primary, secondary, or tertiary structure, as compared to a reference nucleic acid molecule or protein, respectively (e.g., as compared to a wild-type nucleic acid molecule or protein). A variant can be a variant of a full-length Fibcd1 protein or a fragment of Fibcd1.

As used herein, the term “functional derivative of Fibcd1” refers to a protein that is derived from an wild-type or variant Fibcd1 (including fragments) as described herein, which has been chemically modified by techniques such as adding additional amino acid residues (e.g., a fusion protein), adding additional side chains, ubiquitination, labeling, PEGylation (derivatization with polyethylene glycol), and insertion, deletion or substitution of amino acid mimetics and/or unnatural amino acids that do not normally occur in the sequence of wild-type Fibcd1 that is the basis of the derivative. For example, in some embodiments, the invention includes an Fibcd1 protein derivative, wherein Fibcd1 (or variant or fragment) is fused with a label, such as, for example, an epitope, e.g., a FLAG® epitope tag or a V5 epitope or an HA epitope. Such a tag can be useful for, for example, purifying the Fibcd1 protein derivative. The term “derivative” also encompasses a derivatized protein, such as, for example, a protein modified to contain one or more-chemical moieties other than an amino acid. The chemical moiety can be linked covalently to the protein, e.g., via an amino-terminal amino acid residue, a carboxy-terminal amino acid residue, or at an internal amino acid residue. Such modifications include the addition of a protective or capping group on a reactive moiety in the polypeptide, addition of a detectable label, and other changes that do not adversely destroy the activity of the Fibcd1 protein. In some embodiments, an Fibcd1 derivative contains additional chemical moieties not normally a part of the molecule. Such moieties can improve its solubility, absorption, biological half-life, etc. The moieties can alternatively decrease the toxicity of the molecule, or eliminate or attenuate an undesirable side effect of the molecule, etc. Moieties capable of mediating such effects are disclosed, for example, in Remington’s Pharmaceutical Sciences, 18^{th} edition, A.R. Gennaro, Ed., Mack Publ., Easton, PA (1990). A derivative can be a derivative of a full-length Fibcd1 protein or a fragment of Fibcd1.

The Fibcd1 derivative described herein can also include unnatural amino acids or modifications of N— or C—terminal amino acids. Examples of such unnatural or modified amino acids include, but are not limited to, N-alkyl amino acids, lactic acid, 4-hydroxy proline, γ-carboxyglutamate, ε-N,N,N-trimethyllysine, ornithine, ε-N-acetyllysine, O-phosphoserine, N-acetylserine, N-formylmethionine, 3-methylhistidine, 5-hydroxylysine, and δ-N-methylarginine. Other modified amino acids include homocysteine, phosphothreonine, phosphotyrosine, hydroxyproline, gamma-carboxyglutamate, hippuric acid, octahydroindole-2-carboxylic acid, 1,2,3,4,-tetrahydroisoquinoline-3-carboxylic acid, penicillamine (3-mercapto-D-valine), citruline, alpha-methyl-alanine, para-benzoylphenylalanine, para-aminophenylalanine, p-fluorophenylalanine, phenylglycine, propargylglycine, sarcosine, tert-butylglycine, diaminobutyric acid, 7-hydroxy-tetrahydroisoquinoline carboxylic acid, naphthylalanine, biphenylalanine, cyclohexylalanine, aminoisobutyric acid, norvaline, norleucine, tert-leucine, tetrahydroisoquinoline carboxylic acid, pipecolic acid, phenylglycine, homophenylalanine, cyclohexylglycine, dehydroleucine, 2,2-diethylglycine, 1-amino-1-cyclopentanecarboxylic acid, 1-amino-1-cyclohexanecarboxylic acid, amino-benzoic acid, amino-naphthoic acid, gamma-aminobutyric acid, difluorophenylalanine, nipecotic acid, alpha-amino butyric acid, thienyl-alanine, aminovaleric acid, pyroglutaminic acid, alpha-aminoisobutyric acid, gamma-aminobutyric acid, alpha-aminobutyric acid, α-napthyalanine, β-napthyalanine, Ac-β-napthyalanine, 4-halo-Phenyl, 4-pyrolidylalanine, isonipecotic carboxylic acid, and any combinations thereof.

When referring to Fibcd1 derivatives that are fusion proteins, said derivatives are created by joining two protein coding sequences together. The fusion proteins described herein are fusion proteins formed by joining a coding sequence of Fibcd1 or variant or fragment thereof with a coding sequence of a second polypeptide (including a peptide) to form a fusion or chimeric coding sequence such that they constitute a single open-reading frame. The fusion coding sequence, when transcribed and translated, expresses a fusion protein. In other words, a “fusion polypeptide” or “fusion protein” is a recombinant protein of two or more proteins which are joined by a peptide bond.

In some embodiments, the Fibcd1 protein is a fusion protein composed of Fibcd1 protein (or fragment or variant) and a delivery peptide, e.g., a cell-penetrating peptide. Cell-penetrating peptides (CPPs, also known as protein transduction domains, membrane translocating sequences, and Trojan peptides) are short peptides (less than or equal to approximately 40 amino acids), which are able to penetrate a cell membrane to gain access to the interior of a cell. Thus, CPPs can be used to facilitate the transfer of proteins to a muscle cell in vivo. For example, the TAT protein transduction domain (PTD), when attached to recombinant full-length utrophin and micro-utrophin protein, has been able to successfully transfer utrophin proteins to the muscle of mdx mice (Sonnemann, et al. (2009) PLoS Med. 6(5):e1000083).

CPPs that can be used in accordance with the invention include, but are not limited to, Penetratin or Antenapedia PTD

(RQIKWFQNRRMKWKK; SEQ ID NO:4)

, TAT

(YGRKKRRQRRR; SEQ ID NO: 5)

or a modified TAT having one or more mutated residues

(e.g., YARAAARQARA; SEQ ID NO:6)

, R9-Tat

GRRRRRRRRRPPQ; SEQ ID NO: 7)

, R10

(RRRRRRRRRR; SEQ ID NO:8)

, SynB1

(RGGRLSYSRRRFSTSTGR; SEQ ID NO:9)

, SynB3

(RRLSYSRRRF; SEQ ID NO:10)

, PTD-4

(PIRRRKKLRRLK; SEQ ID
NO:11)

, PTD-5

(RRQRRTSKLMKR; SEQ ID NO:12)

, FHV Coat-(35-49)

(RRRRNRTRRNRRRVR; SEQ ID NO: 13)

, BMV Gag-(7-25)

(KMTRAQRRAAARRNRWTAR; SEQ ID NO:14)

, HTLV-II Rex-(4-16)

(TRRQRTRRARRNR; SEQ ID NO:15)

, D-Tat

(GRKKRRQRRRPPQ; SEQ ID NO:16)

, Transportan chimera

(GWTLNSAGYLLGKINLKALAALAKKIL;
SEQ ID NO:17)

, MAP

(KLALKLALKLALALKLA; SEQ ID NO:18)

, SBP

(MGLGLHLLVLAAALQGAWSQPKKKRKV; SEQ ID NO:19)

, FBP

(GALFLGWLGAAGSTMGAWSQPKKKRKV; SEQ ID NO:20)

, MPG

(ac-GALFLGFLGAAGSTMGAWSQPKKKRKV; SEQ ID NO:21)

or any other suitable CPP known to one of ordinary skill in the art.

To facilitate the efficient delivery of an Fibcd1 protein (including fragments and variants) to a muscle cell, the cell penetrating peptide, e.g., the TAT PTD, can be attached to or conjugated to the Fibcd1 protein via a covalent linkage (e.g., via an in-frame fusion). Where the attachment or conjugation involves a covalent linkage, the cell penetrating peptide and the Fibcd1 protein, fragment or variant thereof can be directly coupled to each other or can be coupled via a linker molecule. In some embodiments, a covalent linkage can be between nucleotide molecules. In such embodiments, a nucleotide sequence that encodes the CPP can be operably linked to a nucleotide sequence encoding an Fibcd1 protein, so that when expressed by a vector (e.g., a plasmid or a viral vector), the CPP-Fibcd1 protein is expressed as a single fusion protein. Alternatively, the cell penetrating peptide may be attached to the Fibcd1 protein via a non-covalent linkage (e.g., an interaction that is not covalent in nature and provides force to hold the molecules or parts of molecules together, such as ionic bonds, hydrophobic interactions, hydrogen bonds, van-der-Waals forces, and dipole-dipole bonds).

In some aspects, the Fibcd1 protein qualifies as both a fragment and variant, i.e., the Fibcd1 protein has an amino-terminal deletion and/or a carboxyl-terminal deletion when compared to the full-length protein; and has one or more amino acid or nucleic acid deletions, additions, substitutions or side-chain modifications. In other aspects, the Fibcd1 protein qualifies as both a fragment and derivative, i.e., the Fibcd1 protein has an amino-terminal deletion and/or a carboxyl-terminal deletion when compared to the full-length protein; and has been chemically modified to include additional amino acid residues (e.g., a fusion protein), additional side chains, ubiquitination, labeling, PEGylation, and insertion, deletion or substitution of amino acid mimetics and/or unnatural amino acids that do not normally occur in the sequence of wild-type Fibcd1. In yet other aspects, the Fibcd1 protein qualifies as a fragment, variant and derivative, i.e., the Fibcd1 protein has an amino-terminal deletion and/or a carboxyl-terminal deletion when compared to the full-length protein; has one or more amino acid or nucleic acid deletions, additions, substitutions or side-chain modifications; and has been chemically modified to include additional amino acid residues, additional side chains, ubiquitination, labeling, PEGylation, and insertion, deletion or substitution of amino acid mimetics and/or unnatural amino acids that do not normally occur in the sequence of wild-type Fibcd1. Accordingly, a recombinant Fibcd1 protein of this invention includes a Fibcd1 fragment, a Fibcd1 variant, a Fibcd1 derivative, or a combination thereof.

In one aspect, this invention provides for the delivery of Fibcd1 protein (including a fragment, variant, and/or derivative thereof) to ameliorate or treat myofiber atrophy by, e.g., improving muscle cell size/mass and muscle strength. Administration of Fibcd1 protein can result from delivery of the protein to the cell or by delivery of a polynucleotide or nucleic acid molecule, such as a DNA or RNA, encoding the Fibcd1 protein to a cell, wherein the polynucleotide is transcribed, and the transcript is translated, to generate the Fibcd1 protein. Trans-splicing, polypeptide cleavage and polypeptide ligation can also be involved in expression of an Fibcd1 protein in a cell.

Accordingly, in some aspects, this invention provides for the preparation and administration of Fibcd1 protein to a subject in need of treatment, e.g., a subject having or at risk of experiencing muscle atrophy. The Fibcd1 protein can be obtained by conventional recombinant and/or chemical synthesis methods and includes a fragment, variant, or derivative of Fibcd1 protein. For recombinant production, nucleic acids encoding the Fibcd1 protein are introduced into a host cell (e.g., as an element of a vector as described below), expressed by the host cell, isolated from the cell or cell culture medium and optionally purified. Suitable host cells and vectors for recombinant protein expression are known in the art and available from a number of commercial sources. The term “isolated” refers to a molecule that is substantially separated from its natural environment. For instance, an isolated protein is one that is substantially separated from a cell or tissue source. The term “purified” refers to a molecule that is substantially free of other material that associates with the molecule in its natural environment. For instance, a purified protein is substantially free of the cellular material or other proteins from a cell from which it is derived. The term refers to preparations where the isolated protein is sufficiently pure to be administered as a therapeutic composition, or at least 70% to 80% (w/w) pure, more preferably, at least 80%-90% (w/w) pure, even more preferably, 90-95% pure; and, most preferably, at least 95%, 96%, 97%, 98%, 99%, or 100% (w/w) pure.

In other aspects, this invention provides gene therapy vectors and methods thereof for the in vivo production of a Fibcd1 protein described herein. Such therapies achieve therapeutic effects by introduction of the polynucleotide sequences into cells or tissues in a subject having any of the diseases or conditions described herein. As used herein, the term “vector” refers to a nucleic acid molecule capable of transporting or mediating expression of a heterologous nucleic acid to which it has been linked, i.e., an Fibcd1 protein, to a host cell; a plasmid is a species of the genus encompassed by the term “vector.” The term “vector” typically refers to a nucleic acid sequence containing an origin of replication and other entities necessary for replication and/or maintenance in a host cell. Vectors capable of directing the expression of genes and/or nucleic acid sequence to which they are operatively linked are referred to herein as “expression vectors”. In general, expression vectors are often in the form of “plasmids” which refer to circular double-stranded DNA molecules which, in their vector form are not bound to the chromosome, and typically include entities for stable or transient expression or the encoded DNA. Other expression vectors that can be used in the methods as disclosed herein include, but are not limited to plasmids, episomes, bacterial artificial chromosomes, yeast artificial chromosomes, bacteriophages or viral vectors, and such vectors can integrate into the host’s genome or replicate autonomously in the particular cell. A vector can be a DNA or RNA vector. Other forms of expression vectors known by those skilled in the art, which serve the equivalent functions can also be used, e.g., self-replicating extrachromosomal vectors or vectors that integrate into a host genome. Preferred vectors are those capable of autonomous replication and/or expression of nucleic acids to which they are linked.

In some embodiments, delivery of polynucleotide sequences encoding the Fibcd1 protein described herein can be achieved using a recombinant expression vector. Various viral vectors which can be used for gene therapy include, for example, adenovirus, herpes virus, vaccinia, or an RNA virus such as a retrovirus. The retroviral vector can be a derivative of a murine or avian retrovirus. Such expression methods have been used in gene delivery and are well-known in the art. For example, in regard to muscle-specific gene delivery, US 2011/0212529 describes muscle-specific expression vectors including muscle-specific enhancers and promoter elements derived from a muscle creatine kinase promoter and enhancers, a troponin I promoter and internal regulatory elements, a skeletal alpha-actin promoter, or a desmin promoter and enhancers. See also, e.g., Odom, et al. (2011) Mol. Ther. 19(1):36-45; Percival, et al. (2007) Traffic 8 (10) :1424-39; and Gregorevic, et al. (2006) Nat. Med. 12(7):787-9, which describe, in part, muscle-specific gene therapy methods. See also US 2003/0099671, which discloses a mutated rabies virus suitable for delivering a gene to a subject; US 6,310,196, which describes a DNA construct that is useful for gene therapy; and US 6,140,111, which disclose retroviral vectors suitable for human gene therapy in the treatment of a variety of diseases.

Retroviruses provide a convenient platform for gene delivery. A selected gene can be inserted into a vector and packaged in retroviral particles using techniques known in the art. The recombinant virus can then be isolated and delivered to cells of the subject either in vivo or ex vivo. A number of retroviral systems have been described. See, e.g., US 5,219,740; Miller & Rosman (1989) BioTechniques 7:980-90; Miller (1990) Human Gene Therapy 1:5-14; Scarpa, et al. (1991) Virology 180:849-52; Burns, et al. (1993) Proc. Natl. Acad. Sci. USA 90:8033-37; Boris-Lawrie & Temin (1993) Curr. Opin. Genet. Develop. 3:102-09. Examples of retroviral vectors in which a heterologous gene can be inserted include, but are not limited to: Moloney murine leukemia virus (MoMuLV), Harvey murine sarcoma virus (HaMuSV), murine mammary tumor virus (MuMTV), and Rous sarcoma virus (RSV). A number of additional retroviral vectors can incorporate multiple genes. All of these vectors can transfer or incorporate a gene for a selectable marker so that transduced cells can be identified and generated. By inserting, for example, a polynucleotide sequence encoding an Fibcd1 protein of interest into the viral vector, along with another gene which encodes a ligand for a receptor on a specific target cell, such as, for example, a muscle cell, the vector is now target specific.

Replication-defective murine retroviral vectors are widely used gene transfer vectors. Murine leukemia retroviruses include a single stranded RNA molecule complexed with a nuclear core protein and polymerase (pol) enzymes, encased by a protein core (gag), and surrounded by a glycoprotein envelope (env) that determines host range. The genomic structure of retroviruses includes gag, pol, and env genes and 5′ and 3′ long terminal repeats (LTRs). Retroviral vector systems exploit the fact that a minimal vector containing the 5′ and 3′ LTRs and the packaging signal are sufficient to allow vector packaging, infection and integration into target cells, provided that the viral structural proteins are supplied in trans in the packaging cell line. Fundamental advantages of retroviral vectors for gene transfer include efficient infection and gene expression in most cell types, precise single copy vector integration into target cell chromosomal DNA and ease of manipulation of the retroviral genome.

In some embodiments, a nucleotide sequence encoding an Fibcd1 protein is inserted into an adenovirus-based expression vector. Unlike retroviruses, which integrate into the host genome, adenoviruses persist extrachromosomally thus minimizing the risks associated with insertional mutagenesis (Haj-Ahmad & Graham (1986) J. Virol. 57:267-74; Bett, et al. (1993) J. Virol. 67:5911-21; Mittereder, et al. (1994) Human Gene Therapy 5:717-29; Seth, et al. (1994) J. Virol. 68:933-40; Barr, et al. (1994) Gene Therapy 1:51-58; Berkner (1988) BioTechniques 6:616-29; and Rich, et al. (1993) Human Gene Therapy 4:461-76) .

Adenoviral vectors for use with the present invention can be derived from any of the various adenoviral serotypes, including, without limitation, any of the over 40 serotype strains of adenovirus, such as serotypes 2, 5, 12, 40, and 41. The adenoviral vectors used herein are replication-deficient and contain the gene of interest under the control of a suitable promoter, such as any of the promoters discussed below with reference to adeno-associated virus. For example, US 6,048,551 describes replication-deficient adenoviral vectors that can be used to express a protein of interest under the control of the Rous Sarcoma Virus (RSV) promoter. Other recombinant adenoviruses of various serotypes, and including different promoter systems, can be created by those skilled in the art. See, e.g., US 6,306,652. Moreover, “minimal” adenovirus vectors, as described in US 6,306,652, will find use with the present invention. Other useful adenovirus-based vectors for delivery of an Fibcd1 protein include the “gutless” (helper-dependent) adenovirus in which the vast majority of the viral genome has been removed (Wu, et al. (2001) Anesthes. 94:1119-32).

Another viral system that can be used for gene delivery of the Fibcd1 proteins described herein is Adeno-associated virus (AAV). AAV is a helper-dependent virus; that is, it requires co-infection with a helper virus (e.g., adenovirus, herpesvirus or vaccinia) in order to form AAV virions in the wild. In the absence of co-infection with a helper virus, AAV establishes a latent state in which the viral genome inserts into a host cell chromosome, but infectious virions are not produced. Subsequent infection by a helper virus rescues the integrated genome, allowing it to replicate and package its genome into infectious AAV virions. Recombinant AAV virions including a nucleic acid molecule encoding an Fibcd1 protein can be produced using a variety of art-recognized techniques. In some embodiments, a rAAV vector construct is packaged into rAAV virions in cells co-transfected with wild-type AAV and a helper virus, such as adenovirus. See, e.g., US 5,139,941. Alternatively, plasmids can be used to supply the necessary replicative functions from AAV and/or a helper virus. In some embodiments, rAAV virions are produced using a plasmid to supply necessary AAV replicative functions (the “AAV helper functions”). See, e.g., US 5,622,856 and US 5,139,941. In another embodiment, a triple transfection method is used to produce rAAV virions. See US 6,001,650 and US 6,004,797. Recombinant AAV expression vectors can be constructed using standard techniques of molecular biology. rAAV vectors include a transgene of interest flanked by AAV ITRs at both ends. rAAV vectors are also constructed to contain transcription control elements operably linked to the transgene sequence, including a transcriptional initiation region and a transcriptional termination region. See, e.g., US 5,173,414; US 5,139,941; WO 92/01070; WO 93/03769; Lebkowski, et al. (1988) Molec. Cell. Biol. 8:3988-96; Vincent, et al. (1990) Vaccines 90 (Cold Spring Harbor Laboratory Press); Carter (1992) Curr. Opin. Biotechnol. 3:533-39.

Suitable host cells for producing rAAV virions of the present invention from rAAV expression vectors include microorganisms, yeast cells, insect cells, and mammalian cells. Such host cells are preferably capable of growth in suspension culture, a bioreactor, or the like. The term “host cell” includes the progeny of the original cell that has been transfected with an rAAV virion. Cells from the stable human cell line, 293 (readily available through the American Type Culture Collection under Accession Number ATCC CRL1573) are an example of a cell line of use in the practice of the present invention. The human cell line 293 is a human embryonic kidney cell line that has been transformed with adenovirus type-5 DNA fragments (Graham, et al. (1977) J. Gen. Virol. 36:59), and expresses the adenoviral E1a and E1b genes (Aiello, et al. (1979) Virology 94:460). The 293 cell line is readily transfected, and provides a particularly convenient platform in which to produce rAAV virions.

Additional viral vectors useful for delivering the nucleic acid molecules and/or expressing an Fibcd1 protein include those derived from the pox family of viruses, including vaccinia virus and avian poxvirus. By way of example, vaccinia virus recombinants expressing an Fibcd1 protein can be constructed as follows. DNA carrying the Fibcd1 protein is inserted into an appropriate vector adjacent to a vaccinia promoter and flanking vaccinia DNA sequences, such as the sequence encoding thymidine kinase (TK). This vector is then used to transfect cells that are simultaneously infected with vaccinia. Homologous recombination serves to insert the vaccinia promoter and the gene into the viral genome. The resulting TK-recombinant can be selected by culturing the cells in the presence of 5-bromodeoxyuridine and picking viral plaques resistant thereto.

Alternatively, avipoxviruses, such as the fowlpox and canarypox viruses, can be used to express an Fibcd1 protein. Recombinant avipox viruses expressing immunogens from mammalian pathogens are known to confer protective immunity when administered to non-avian species. The use of avipox vectors in human and other mammalian species is advantageous with regard to safety because members of the avipox genus can only productively replicate in susceptible avian species. Methods for producing recombinant avipoxviruses are known in the art and employ genetic recombination, as described above with respect to the production of vaccinia viruses. See, e.g., WO 91/12882; WO 89/03429; and WO 92/03545.

Molecular conjugate vectors, such as the adenovirus chimeric vectors, can also be used for gene delivery. Michael, et al. (1993) J. Biol. Chem. 268:6866-69 and Wagner, et al. (1992) Proc. Natl. Acad. Sci. USA 89:6099-6103. Members of the Alphavirus genus, for example the Sindbis and Semliki Forest viruses, may also be used as viral vectors for delivering and expressing an Fibcd1 protein. See, e.g., Dubensky, et al. (1996) J. Virol. 70:508-19; WO 95/07995; WO 96/17072.

Another targeted delivery system for a nucleic acid molecule encoding an Fibcd1 protein is a colloidal dispersion system. Colloidal dispersion systems include, for example, macromolecule complexes, nanocapsules, microspheres, beads, and lipid-based systems including oil-in-water emulsions, micelles, mixed micelles, and liposomes. Liposomes are artificial membrane vesicles which are useful as delivery vehicles in vitro and in vivo. RNA, DNA and intact virions can be encapsulated within the aqueous interior and be delivered to cells in a biologically active form (see, for example, Fraley, et al. (1981) Trends Biochem. Sci. 6:77). Methods for efficient gene transfer using a liposome vehicle, are known in the art (see, for example, Mannino, et al. (1988) Biotechniques 6:682. The composition of the liposome is usually a combination of phospholipids, usually in combination with steroids, especially cholesterol. Other phospholipids or other lipids may also be used. The physical characteristics of liposomes depend on pH, ionic strength, and the presence of divalent cations. Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Illustrative phospholipids include egg phosphatidylcholine, dipalmitoylphosphatidylcholine and distearoylphosphatidylcholine. The targeting of liposomes is also possible based on, for example, organ-specificity, cell-specificity, and organelle-specificity and is known in the art.

In some embodiments, cells expressing an Fibcd1 protein can be delivered by direct application, for example, direct injection of a sample of such cells into a target site, such as muscle tissue thereby delivering the Fibcd1 protein. These cells can be purified. In some embodiments, such cells can be delivered in a medium or matrix which partially impedes their mobility so as to localize the cells to a target site. Such a medium or matrix could be semi-solid, such as a paste or gel, including a gel-like polymer. Alternatively, in some embodiments, the medium or matrix could be in the form of a solid, a porous solid which will allow the migration of cells into the solid matrix, and hold them there while allowing proliferation of the cells.

In other embodiments, an Fibcd1 protein is delivered to a cell or administered to a subject in the form of a modified RNA encoding the Fibcd1 protein. A modified RNA encoding the Fibcd1 protein described herein can include a modification to prevent rapid degradation by endo- and exo-nucleases and/or to avoid or reduce the cell’s innate immune or interferon response to the RNA. Modifications include, but are not limited to, e.g., (a) end modifications such as 5′-end modifications (phosphorylation dephosphorylation, conjugation, inverted linkages, etc.) and 3′-end modifications (conjugation, DNA nucleotides, inverted linkages, etc.); (b) base modifications, e.g., replacement with modified bases, stabilizing bases, destabilizing bases, or bases that base pair with an expanded repertoire of partners, or conjugated bases; (c) sugar modifications (e.g., at the 2′ position or 4′ position) or replacement of the sugar; and/or (d) internucleoside linkage modifications, including modification or replacement of the phosphodiester linkages. To the extent that such modifications interfere with translation (i.e., results in a reduction of 50% or more in translation relative to the lack of the modification (e.g., in a rabbit reticulocyte in vitro translation assay), the modification is not suitable for the methods and compositions described herein. Specific examples of modified RNA compositions useful with the methods described herein include, but are not limited to, RNA molecules containing modified or non-natural internucleoside linkages. Modified RNAs having modified internucleoside linkages include, among others, those that do not have a phosphorus atom in the internucleoside linkage. In other embodiments, the modified RNA has a phosphorus atom in its internucleoside linkage(s). Non-limiting examples of modified internucleoside linkages include phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included.

Modified internucleoside linkages that do not include a phosphorus atom therein have internucleoside linkages that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Another modification for use with the synthetic, modified RNA described herein involves chemically linking to the RNA one or more ligands, moieties or conjugates that enhance the activity, cellular distribution or cellular uptake of the RNA. Ligands can be particularly useful where, for example, a synthetic, modified RNA is administered in vivo. Such moieties include but are not limited to lipid moieties such as a cholesterol moiety (Letsinger, et al. (1989) Proc. Natl. Acid. Sci. USA 86:6553-6556), cholic acid (Manoharan, et al. (1994) Biorg. Med. Chem. Let. 4:1053-1060), a thioether, e.g., beryl-S-tritylthiol (Manoharan, et al. (1992) Ann. NY Acad. Sci. 660:306-309; Manoharan, et al. (1993) Biorg. Med. Chem. Lett. 3:2765-2770), a thiocholesterol (Oberhauser, et al. (1992) Nucl. Acids Res. 20:533-538), an aliphatic chain, e.g., dodecandiol or undecyl residues (Saison-Behmoaras, et al. (1991) EMBO J. 10:1111-1118; Kabanov, et al. (1990) FEBS Lett. 259:327-330; Svinarchuk, et al. (1993) Biochimie 75:49-54), a phospholipid, e.g., di-hexadecyl-rac-glycerol or triethyl-ammonium 1,2-di-O-hexadecyl-rac-glycero-3-phosphonate (Manoharan, et al. (1995) Tetrahedron Lett. 36:3651-3654; Shea, et al. (1990) Nucl. Acids Res. 18:3777-3783), a polyamine or a polyethylene glycol chain (Manoharan, et al. (1995) Nucleosides & Nucleotides 14:969-973), adamantane acetic acid (Manoharan, et al. (1995) Tetrahedron Lett. 36:3651-3654), a palmityl moiety (Mishra, et al. (1995) Biochim. Biophys. Acta 1264:229-237), or an octadecylamine or hexylamino-carbonyloxycholesterol moiety (Crooke, et al. (1996) J. Pharmacol. Exp. Ther. 277:923-937) .

The modified RNA encoding a Fibcd1 protein described herein can further include (i) a 5′ cap, e.g., 5′ diguanosine cap, tetraphosphate cap analogs having a methylene-bis(phosphonate) moiety, dinucleotide cap analogs having a phosphorothioate modification, cap analogs having a sulfur substitution for a non-bridging oxygen, N7-benzylated dinucleoside tetraphosphate analogs, or anti-reverse cap analogs; (ii) a 5′ and/or 3′ untranslated region (UTR), e.g., a UTR from an mRNA known to have high stability in the cell (e.g., a murine alpha-globin 3′ UTR); (iii) a Kozak sequence; and/or (iv) a poly (A) tail of, e.g., at least 5 adenine nucleotides in length and can be up to several hundred adenine nucleotides.

A nucleic acid molecule encoding an Fibcd1 protein (e.g., DNA vector or RNA) can be introduced into a cell in any manner that achieves intracellular delivery of the nucleic acid molecule, such that expression of the polypeptide encoded by the nucleic acid molecule can occur. As used herein, the term “transfecting a cell” refers to the process of introducing nucleic acids into cells using means for facilitating or effecting uptake or absorption into the cell, as is understood by those skilled in the art. Absorption or uptake of a nucleic acid molecule can occur through unaided diffusive or active cellular processes, or by auxiliary agents or devices. Exemplary methods for introducing a nucleic acid molecule into a cell include, for example, transfection, nucleofection, lipofection, electroporation (see, e.g., Wong & Neumann, (1982) Biochem. Biophys. Res. Commun. 107:584-87), microinjection (e.g., by direct injection of the nucleic acid molecule), biolistics, cell fusion, and the like. In an alternative embodiment, a nucleic acid molecule can be delivered using a drug delivery system such as a nanoparticle, a dendrimer, a polymer, a liposome, or a cationic delivery system. Positively charged cationic delivery systems facilitate binding of a nucleic acid molecule (negatively charged polynucleotides) and also enhances interactions at the negatively charged cell membrane to permit efficient cellular uptake. Cationic lipids, dendrimers, or polymers can either be bound to the nucleic acid molecule, or induced to form a vesicle or micelle (see e.g., Kim, et al. (2008) J. Contr. Rel. 129(2):107-116) that encases the nucleic acid molecule.

In embodiments involving in vivo administration of a nucleic acid molecule encoding an Fibcd1 protein or compositions thereof, the nucleic acid molecule is formulated in conjunction with one or more penetration enhancers, surfactants and/or chelators. Suitable surfactants include fatty acids and/or esters or salts thereof, bile acids and/or salts thereof. Suitable bile acids/salts include chenodeoxycholic acid (CDCA) and ursodeoxychenodeoxycholic acid (UDCA), cholic acid, dehydrocholic acid, deoxycholic acid, glucholic acid, glycholic acid, glycodeoxycholic acid, taurocholic acid, taurodeoxycholic acid, sodium tauro-24,25-dihydro-fusidate and sodium glycodihydrofusidate. Suitable fatty acids include arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof (e.g., sodium). In some embodiments, combinations of penetration enhancers are used, for example, fatty acids/salts in combination with bile acids/salts. One exemplary combination is the sodium salt of lauric acid, capric acid and UDCA. Further penetration enhancers include polyoxyethylene-9-lauryl ether, polyoxyethylene-20-cetyl ether.

For administration to a subject in need thereof, e.g., a subject diagnosed with or predisposed or at risk of muscle atrophy, the Fibcd1 protein and/or expression vector and/or modified RNA encoding the Fibcd1 protein can be provided in a pharmaceutically acceptable composition. As used herein, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The pharmaceutically acceptable composition can further include one or more pharmaceutically carriers (additives) and/or diluents. As used herein, the term “pharmaceutically acceptable carrier” means a pharmaceutically acceptable material, composition or vehicle, such as a liquid, diluent, excipient, manufacturing aid or encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include, but are not limited to, gelatin, buffering agents, such as magnesium hydroxide and aluminum hydroxide, pyrogen-free water, isotonic saline, Ringer’s solution, pH buffered solutions, bulking agents such as polypeptides and amino acids, serum component such as serum albumin, HDL and LDL, and other non-toxic compatible substances employed in pharmaceutical formulations. Preservatives and antioxidants can also be present in the formulation. The terms such as “excipient”, “carrier”, “pharmaceutically acceptable carrier” or the like are used interchangeably herein.

Pharmaceutically acceptable carriers can vary in a composition of the invention, depending on the administration route and formulation. For example, the pharmaceutically acceptable composition of the invention can be delivered via injection. These routes for administration (delivery) include, but are not limited to, subcutaneous or parenteral including intravenous, intracortical, intracranial, intramuscular, intraperitoneal, and infusion techniques. In another embodiment, the pharmaceutical composition is formulated for intramuscular injection.

The Fibcd1 protein and/or the composition thereof can be formulated in pharmaceutically acceptable compositions which include a therapeutically effective amount of the protein, formulated together with one or more pharmaceutically acceptable carriers (additives) and/or diluents. The protein can be specially formulated for administration in solid or liquid form, including those adapted for the following: (1) parenteral administration, for example, by subcutaneous, intramuscular, intravenous or epidural injection as, for example, a sterile solution or suspension, or sustained-release formulation; (2) topical application, for example, as a cream, ointment, or a controlled-release patch or spray applied to the skin; (3) intravaginally or intrarectally, for example, as a pessary, cream or foam; (4) sublingually; (5) ocularly; (6) transdermally; (7) transmucosally; or (8) nasally. Additionally, the protein can be implanted into a patient or injected using a drug delivery system. See, for example, Urquhart, et al. (1984) Ann. Rev. Pharmacol. Toxicol. 24:199-236; US 3,773,919; or US 3,270,960.

When administering a pharmaceutical composition of the invention parenterally, it will be generally formulated in a unit dosage injectable form (solution, suspension, emulsion). The pharmaceutical formulations suitable for injection include sterile aqueous solutions or dispersions. The carrier can be a solvent or dispersing medium containing, for example, water, cell culture medium, buffers (e.g., phosphate-buffered saline), polyol (for example, glycerol, propylene glycol, liquid polyethylene glycol, and the like), and suitable mixtures thereof. In some embodiments, the pharmaceutical carrier can be a buffered solution (e.g., phosphate-buffered saline).

In some embodiments, the pharmaceutical composition can be formulated in an emulsion or a gel. In such embodiments, at least one Fibcd1 protein or vector encoding a Fibcd1 protein or modified RNA encoding an Fibcd1 protein can be encapsulated within a biocompatible gel, e.g., hydrogel and a peptide gel. The gel pharmaceutical composition can be implanted into the muscle or tissue proximal thereto.

Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it may be desirable to include isotonic agents, for example, sugars, sodium chloride, and the like.

The compositions can also contain auxiliary substances such as wetting or emulsifying agents, pH buffering agents, gelling or viscosity enhancing additives, preservatives, colors, and the like, depending upon the route of administration and the preparation desired. Standard texts, such as Remington’s Pharmaceutical Sciences, 18^{th} edition, A.R. Gennaro, Ed., Mack Publ., Easton, PA (1990), may be consulted to prepare suitable preparations, without undue experimentation. With respect to compositions of the invention, however, any vehicle, diluent, or additive used should have to be biocompatible or inert with the Fibcd1 protein or a vector encoding the Fibcd1 protein or modified RNA encoding the Fibcd1 protein.

The compositions can be isotonic, i.e., they can have the same osmotic pressure as blood. The desired isotonicity of the compositions of the invention can be accomplished using sodium chloride, or other pharmaceutically acceptable agents such as dextrose, boric acid, sodium tartrate, propylene glycol or other inorganic or organic solutes. In one embodiment, sodium chloride is used in buffers containing sodium ions.

Viscosity of the compositions can be maintained at the selected level using a pharmaceutically acceptable thickening agent. In one embodiment, methylcellulose is used because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the agent selected. The important point is to use an amount which will achieve the selected viscosity. Viscous compositions are normally prepared from solutions by the addition of such thickening agents.

In some embodiment, muscle cells transduced with a vector encoding an Fibcd1 protein can be included in the compositions and stored frozen. In such embodiments, an additive or preservative known for freezing cells can be included in the compositions. A suitable concentration of the preservative can vary from 0.02% to 2% based on the total weight although there may be appreciable variation depending upon the preservative or additive selected. One example of such additive or preservative can be dimethyl sulfoxide (DMSO) or any other cell-freezing agent known to a skilled artisan. In such embodiments, the composition will be thawed before use or administration to a subject, e.g., muscle cell therapy.

Typically, any additives (in addition to the active Fibcd1 protein) can be present in an amount of 0.001 to 50 wt % solution in phosphate buffered saline, and the active ingredient is present in the order of micrograms to milligrams, such as about 0.0001 to about 5 wt %, about 0.0001 to about 1 wt %, about 0.0001 to about 0.05 wt % or about 0.001 to about 20 wt %, about 0.01 to about 10 wt %, and about 0.05 to about 5 wt %. For any therapeutic composition to be administered to a subject in need thereof, and for any particular method of administration, it is preferred to determine toxicity, such as by determining the lethal dose (LD) and LD₅₀ in a suitable animal model, e.g., rodent such as mouse; and, the dosage of the composition(s), concentration of components therein and timing of administering the composition(s), which elicit a suitable response. Such determinations do not require undue experimentation from the knowledge of the skilled artisan.

The compositions of the invention can be prepared by mixing the ingredients following generally accepted procedures. For example, an effective amount of an Fibcd1 protein or vectors encoding an Fibcd1 protein can be resuspended in an appropriate pharmaceutically acceptable carrier and the mixture can be adjusted to the final concentration and viscosity by the addition of water or thickening agent and possibly a buffer to control pH or an additional solute to control tonicity. An effective amount of an Fibcd1 protein described herein and any other additional agent can be mixed with the cell mixture. Generally, the pH can vary from about 3 to about 7.5. In some embodiments, the pH of the composition can be about 6.5 to about 7.5. Compositions can be administered in dosages and by techniques well-known to those skilled in the medical and veterinary arts taking into consideration such factors as the age, sex, weight, and condition of the particular patient, and the composition form used for administration (e.g., liquid). Dosages for humans or other mammals can be determined without undue experimentation by a skilled artisan.

Suitable regimes for initial administration and further doses or for sequential administrations can be varied. In one embodiment, a therapeutic regimen includes an initial administration followed by subsequent administrations, if necessary. In some embodiments, multiple administrations of an Fibcd1 protein can be injected into the subject. For example, an Fibcd1 protein can be administered in two or more, three or more, four or more, five or more, or six or more injections. In some embodiments, the same Fibcd1 protein can be administered in each subsequent administration. In some embodiments, a different Fibcd1 protein described herein can be administered in each subsequent administration.

The subsequent injection can be administered immediately after the previous injection, or after at least about 1 minute, after at least about 2 minute, at least about 5 minutes, at least about 15 minutes, at least about 30 minutes, at least about 1 hour, at least about 2 hours, at least about 3 hours, at least about 6 hours, at least about 12 hours, at least about 24 hours, at least about 2 days, at least about 3 days, at least about 4 days, at least about 5 days, at least about 6 days or at least about 7 days. In some embodiments, the subsequent injection can be administered after at least about 1 week, at least about 2 weeks, at least about 1 month, at least about 2 years, at least about 3 years, at least about 6 years, or at least about 10 years.

In various embodiments, a dosage of a composition described herein is considered to be pharmaceutically effective if the dosage reduces the degree of muscle atrophy, e.g., indicated by changes in muscular morphologies, improvement in muscle size/mass, and/or improvement in muscle function, by at least about 5%, at least about 10%, at least about 15%, at least about 20%, at least about 30%, at least about 40%, or at least about 50%. In some embodiments, the muscle size/mass is improved by more than 50%, e.g., at least about 60%, or at least about 70%. In some embodiments, the muscle size/mass is improved by at least about 80%, at least about 90% or greater, as compared to a control (e.g., in the absence of the composition described herein).

Cells to which the vectors or modified RNAs encoding Fibcd1 proteins may be delivered or administered include, for example, muscle cells, myoblasts, muscle progenitor cells, and stem cells, including pluripotent and multipotent stem cells.

As demonstrated herein, Fibcd1 proteins are useful in pharmaceutical compositions and methods for treating muscle atrophy. In some aspects, the present disclosure is directed to methods of treating muscle atrophy, in particular muscle atrophy associated with aging, injury, disuse, cachexia, nutritional or metabolic derangements, vascular insufficiency, administration of myotoxic xenobiotics and neuromuscular disorders, such as muscular dystrophy, myopathy, and amyotrophic lateral sclerosis. In some embodiments, the method of the invention involves administering to a subject having or at risk for muscle atrophy an effective amount of an Fibcd1 protein, or a fragment, variant, or derivative thereof. In other embodiments of this method and all such methods described herein, the Fibcd1 protein or derivative thereof is administered to the subject in the form of a vector or nucleic acid encoding the Fibcd1 protein or fragment, variant, or derivative thereof.

For the purposes of this invention, the term “treatment” refers to both therapeutic treatment and prophylactic/preventative measures. Those in need of treatment may include individuals already having a particular medical disorder, as well as those who may ultimately acquire the disorder (i.e., those needing preventive measures). A “subject” or “individual” that may be treated in accordance with the method herein is preferably an animal, for example a human or non-human animal. The term “non-human animal” or “non-human mammal” include, for example, mammals such as rats, mice, rabbits, sheep, cats, dogs, cows, pigs, horses and non-human primates.

As indicated, a subject’s muscle atrophy may be the result of aging, injury, disuse (e.g., bed rest or a cast-immobilized limb), cachexia, nutritional or metabolic derangements, vascular insufficiency, drug treatment (e.g., corticosteroid treatment) or a neuromuscular disorder or disease. For example, muscle atrophy can be the result of a disorder or condition such as, for example, cancer cachexia, AIDS cachexia, or cardiac cachexia. Cachexia is generally associated with the massive loss (up to 30% of total body weight) of both adipose tissue and skeletal muscle mass that may occur as a side effect of many diseases such as cancer, AIDS, and chronic heart failure. Muscle atrophy can also be induced by the loss of innervation or damage to innervation of the muscle tissue. Specifically, diseases such as chronic neuropathy and motor neuron disease can cause damage to innervation. Moreover, many times a physical injury to the nerve can lead to damage to the innervation of the muscle tissue. Alternatively, muscle atrophy can be the result of environmental conditions such as during spaceflight or as a result of aging or extended bed rest. Under these environmental conditions, the muscles do not bear the usual weight load, resulting in muscle atrophy from disuse.

Neuromuscular disorders or diseases refer to those disorders in which muscle function in impaired, either directly due to pathologies of the muscle (myopathic disorders), and/or indirectly, due to pathologies of nerves or neuromuscular junctions (neuropathic disorders), and include muscular dystrophies such as severe or benign K-linked muscular dystrophy, limb-girdle dystrophy, facioscapulohumeral dystrophy, myotonic dystrophy, distal muscular dystrophy, progressive dystrophic ophthalmoplegia, oculopharyngeal dystrophy, Duchenne’s muscular dystrophy, and Fakuyama-type congenital muscular dystrophy; polymyositis; amyotrophic lateral sclerosis (ALS); organ atrophy; frailty; carpal tunnel syndrome; congestive obstructive pulmonary disease; congenital myopathy; myotonia congenital; familial periodic paralysis; paroxysmal myoglobinuria; myasthenia gravis; Eaton-Lambert syndrome; secondary myasthenia; cerebrovascular accidents (stroke), Parkinson’s disease, multiple sclerosis, Huntington’s disease (Huntington’s chorea); Creutzfeldt Jakob disease; and sarcopenia, and other muscle wasting syndromes.

The compositions and methods of this invention are also of use in the treatment of muscle atrophy associated with an inflammatory myopathy. Typically, inflammatory myopathies are believed to result from an autoimmune reaction, whereby the body’s own immune system attacks the muscle cells. Examples of inflammatory myopathies include polymyositis and dermatomyositis.

The determination as to whether a subject has a muscle atrophy or a disease or condition that induces muscle atrophy can be made by any measure accepted and utilized by those skilled in the art. For example, diagnosis of subjects with muscular dystrophy is generally contingent on a targeted medical history and examination, biochemical assessment, muscle biopsy, or genetic testing.

The “effective amount” of an Fibcd1 protein or fragment, variant, or derivative thereof described herein is the minimum amount necessary to, for example, increase or improve one or more muscle function parameters, such as, for example, morphology, size/mass, and contractility, as assayed by methods known in the art and described herein. Accordingly, the “effective amount” to be administered to a subject is governed by such considerations, and refers to the minimum amount necessary to prevent, ameliorate, treat, or stabilize, a subject’s muscle atrophy.

In some embodiments, the effective amount is sufficient to reduce muscle atrophy that occurs in muscle cells. Various established in vitro and in vivo assays can be used to determine an effective amount of the Fibcd1 protein or fragment, variant, or derivative thereof for inhibiting muscle atrophy of muscle cells, as described, for example, in the Examples. Exemplary measurable responses are muscle contractility and/or muscle size/mass. Exemplary assays to measure the biological activity of a Fibcd1 protein include, for example, in situ analysis of skeletal muscle contractile function, resistance to exercise-induced fatigue, resistance to stretch contraction-induced injury, and in vitro analysis of diaphragm muscle function. Accordingly, in some embodiments, the effective amount of the Fibcd1 protein is sufficient to increase muscle contraction and/or muscle size/mass by at least about 5%, e.g., by at least about 10%, at least about 20%, at least about 30%, at least about 40%, at least about 50%, at least about 60%, at least about 70%, at least about 80%, at least about 90%, at least about 95%, at least about 96%, about 98%, about 99%, or 100%, as compared to the absence of the Fibcd1 protein.

In some embodiments, the effective amount of an Fibcd1 protein is about 0.1 mg/kg to about 100 mg/kg. In some embodiments, the effective amount of an Fibcd1 protein can be present in an amount of about 0.5 mg/kg to about 100 mg/kg, about 1 mg/kg to about 75 mg/kg, about 3 mg/kg to about 50 mg/kg, about 5 mg/kg to about 25 mg/kg, or about 5 mg/kg to about 15 mg/kg. In some embodiments, the effective amount of an Fibcd1 protein is about 10 mg/kg.

In some embodiments, the effective amount of an Fibcd1 protein is about 5 nM to about 1 M. In some embodiments, the effective amount of an Fibcd1 protein can be present in an amount of about 5 nM to about 5 µM, about 5 nM to about 100 µM, about 5 nM to about 500 µM, about 5 µM to about 1 mM, or about 1 mM to about 1 M.

The following non-limiting examples are provided to further illustrate the present invention.

### Example 1: Materials and Methods

Drosophila Larval Screening and Body Wall Skeletal Muscle Analysis. Flies were maintained at 25° C. with 60% humidity in tubes containing cornmeal/soy flour/yeast fly food. Fly stocks were obtained from either the Bloomington Drosophila Stock Center (BDSC), The National Institute of Genetics Fly Stocks (NIG-Fly), or the Vienna Drosophila Resource Center (VDRC).

To the define the list of Drosophila stocks for transgenic RNAi and overexpression of evolutionary conserved myokines, the following procedures were used: first, a list of 788 predicted secreted factors was filtered to retain only 274 Drosophila secreted factors with sufficient homology to humans, i.e., with a score ≥2 as defined by DIOPT (Hu, et al. (2011) BMC Bioinformatics 12:357); second, the list was further filtered to retain only 111 Drosophila secreted factors with sufficient skeletal muscle expression, as defined by FPKM≥4; lastly, the function of these evolutionary-conserved muscle-secreted factors was screened with 508 stocks for RNAi and overexpression.

For the screen, similar procedures were used as previously described (Hunt, et al. (2019) Cell Reports 28:1268-1281 e1266). Specifically, virgin Mef2-Gal4 females (10 flies for each cross) were mated with males (5 flies for each cross) of each RNAi or overexpression stock and transferred to new food tubes every 3 days to avoid overcrowding. Breeders were maintained at 25° C. The sizes of 3^{rd} instar larvae were assessed qualitatively as either bigger or smaller than control larvae. As secondary validation, skeletal muscles from selected candidates were analyzed by dissecting larvae into filets and by exposing the ventral lateral muscles VL3 and VL4 (each composed of a single myofiber) from abdominal segments 2-4, which were then fixed for 30 minutes with 4% EM-grade paraformaldehyde in phosphate-buffered saline (PBS) without Ca²⁺ and Mg²⁺ (Demontis & Perrimon (2009) Development 136:983-993). Following washes, larval body wall muscles (typically from 10 larvae) were stained with DAPI and imaged to detect the endogenous fluorescence of a Mhc-GFP fusion protein. Subsequently, the total area, width, and length of VL3+VL4 myofibers was quantified with the Zeiss Zen software.

Cell Culture. C2C12 mouse muscle cells, mouse Lewis lung carcinoma cells (LLC; Kaplan, et al. (2005) Nature 438:820-827), human embryonic kidney cells (HEK293T), mouse breast cancer cells (4T1; Jia, et al. (2019) EMBO J. 38:e101302; Labelle, et al. (2014) Proc. Natl. Acad. Sci. USA 111:E3053-3061), and human bone osteosarcoma cells (Saos-2; Yu, et al. (2017) BMC Cancer 17:78) were obtained from the ATCC and screened regularly to ensure the absence of mycoplasma (Universal Mycoplasma detection kit). C2C12, LLC, and HEK293 cells were cultured at 37° C. with 5% CO₂ in DMEM (high glucose DMEM, with GlutaMAX™, GIBCO) containing 10% fetal bovine serum (GIBCO), and penicillin/streptomycin (10,000 U/ML, GIBCO). 4T1 cells were maintained at 37° C. with CO₂ in RPMI-1640 (ATCC) containing 10% of fetal bovine serum (ATCC) whereas Saos-2 cells line were cultured in McCoy’s medium (ATCC) containing 10% of fetal bovine serum (ATCC).

Production of Recombinant Mouse Fibcd1. The ~38-kDa C-terminal part of mouse Fibcd1 (composed of 282 amino acids, approximately corresponding to the secreted Fibcd1 fragment retrieved from the cell culture medium) was cloned into the pAcGP67-B vector with BamHI and BglII. Expression of the C-terminal part of mouse Fibcd1 was achieved with the Bac-to-Bac™ Baculovirus Expression System (Invitrogen) and the recombinant protein (rFibcd1) retrieved in PBS (GIBCO) with 1% bovine serum albumin (BSA). The identity of purified rFibcd1 was confirmed by mass-spectrometry.

C2C12 Myotube siRNA Transfection and rFibcdl Treatment. C2C12 cells were maintained as myoblasts with media containing 10% fetal bovine serum and switched to 2% horse serum-containing media (GIBCO) to induce differentiation into myotubes when near confluence. Four days after differentiation, myotube-enriched cultures were generated by adding media containing 4 µg/mL of Cytosine β-D-arabinofuranoside (Ara-C, Sigma) for a further 2 days at which point the remaining myotubes were transfected with siRNAs. To this purpose, myotubes were transfected with 50 µM siRNAs targeting the specified gene or with control non-targeting (NT) siRNAs, by using a ratio of 2 µL Lipofectamine™ 2000 (Invitrogen) to 50 pmol of siRNA in OptiMEM™ (GIBCO), as previously done (Hunt, et al. (2019) Cell reports 28:1268-1281 e1266). Myotube size was assayed 2 days after transfection. ON-TARGET plus siRNA reagents (Dharmacon) used are the following: mouse Fibcd1, non-targeting (NT) control, Wnt9a, Tgfbi, Bmp1, and Sparc.

For concurrent treatment with rFibcd1, myotubes were then treated for 24 hours with either serum-free media containing 100 ng/mL rFibcd1 or control serum-free media (containing the same amount of BSA as the medium with rFibcd1). Myotubes were then fixed and stained for myosin heavy chain (MF20 clone, eBioscience) and myotube diameters analyzed by ImageJ, as explained in detailed below. For the analysis of myoblast fusion, siRNAs were transfected into myoblasts following procedures as explained above.

Nutrient-Starvation Experiments. For nutrient starvation experiments, C2C12 myotubes cells were transfected as indicated above with Fibcd1 or NT siRNAs. After 48 hours, the normal cell culture media (10% FBS in DMEM-high glucose) was replaced with culture media diluted 1:10 in Dulbecco’s PBS for 8 hours and 24 hours (Stevenson, et al. (2005) J. Appl. Physiol. 98:1396-1406).

Western Blotting. For cell culture experiments, the cells were homogenized in 100 µL of NP40 cell lysis buffer (Invitrogen). Subsequently, the cell samples were sonicated for 10 seconds at 30% of frequency, and the protein extracts were quantified by using Bradford assay (Bio-Rad Protein assay dye reagent concentrate). Protein samples were prepared by addition of SDS-Blue loading buffer (Cell Signaling) and DTT (dithiothreitol; to a final concentration of 0.1 M, Cell Signaling) and heating at 95° C. for 5 minutes. Samples were then run on 4-20% gradient SDS-PAGE gels (Mini-PROTEAN® TGX Pre-cast gels, Bio-Rad) alongside a molecular weight ladder (Precision Plus protein standard, Bio-Rad) and transferred to PVDF membranes (IMMOBILON®-P PVDF membrane, Millipore), which were blocked with either 5% milk powder or 5% BSA (according to manufacturer’s instructions) for 1 hour.

Subsequently, the membranes were incubated overnight at 4° C. with the following primary antibodies: mouse anti-phospho-p44/42 MAPK (Erk1/2; Thr202/Tyr204) (Cell Signaling), rabbit anti-phospho-p38 MAPK (Thr180/Tyr182) (Cell Signaling), rabbit anti-phospho-Smad2 (Ser465/467) (Cell Signaling), rabbit anti-phospho-Akt (Ser473; D9e) (Cell Signaling), rabbit anti-phospho-SAPK/JNK (Thr183/Tyr185; 81E11) (Cell Signaling), rabbit anti-α-Tubulin (11h10) (Cell Signaling), rabbit anti-SQSTM1/p62 (Cell Signaling), and mouse anti-FLAG (M2) (Sigma). After washing, the appropriate horseradish peroxidase (HRP)-conjugated secondary antibodies were incubated at 4° C. for 2 hours, washed again, and then probed with ECL reagents (Amersham ECL Western Blotting Detection Reagents) to detect the protein of interest. Ponceau S (Sigma) staining was also used to confirm even loading of protein samples.

Pharmacological Inhibition of ERK. C2C12 myotubes were transfected individually with rFibcd1 or NT siRNA for 48 hours, as explained in detail above. The cells were incubated in serum-free media overnight and they were then treated at the same time with either control serum-free media or serum-free media containing rFibcd1 at 100 ng/mL, and/or Pyrazolylpyrrole (pharmacological inhibitor o+f ERK; Santa Cruz; Hunt, et al. (2015) Genes Dev. 29:2475-2489; Aronov, et al. (2007) J. Med. Chem. 50:1280-1287; Junttila, et al. (2008) FASEB J. 22:954-965) at 2.5 ng/mL, for a further 24 hours. Cells were then fixed and stained with anti-Myosin Heavy Chain antibodies (MF20 clone, eBioscience) and myotube size determined with ImageJ.

C2C12 Myotube Size Analysis. To determine the myotube size, cultures of differentiated C2C12 myotubes were fixed by adding an equal volume of 4% PFA (Electron Microscopy Sciences) to the medium for 10 minutes. Cells were then washed with PBS and blocked for 1 hour in blocking buffer containing PBS with 0.1% Triton™ X-100 and 2% BSA, and then incubated with anti-myosin heavy chain antibodies (MF20 clone, eBioscience) overnight at 4° C. The cells were then stained with a fluorophore-conjugated anti-mouse secondary antibody to detect myosin heavy chain and with DAPI (Roche), to visualize the myotube area and the nuclei, respectively. The size of myotubes was measured by taking the average width value across a myotube at three separate points along it. Typically, ~100 myotubes were measured for each group, as previously described (Hunt, et al. (2019) Cell reports 28:1268-1281 e1266).

HEK293 Transfection. HEK293 cells were transfected with either empty vector (EV) or vector containing C-terminal FLAG-tagged full length (FL) or short (SH) Fibcd1. Two days after transfection, growth media was replaced with serum-free media for an additional 24 hours. The media was then collected alongside the cell lysate using NP40 buffer and the samples run on SDS-PAGE and analyzed by western blot with anti-FLAG antibodies (Sigma clone M2).

Treatment of LLC, 4T1, and Sao-2 Cancer Cells with rFibcdl. The cells were incubated in serum-free media overnight and they were then treated with rFibcd1 at 10 ng/mL and 100 ng/mL for 30 and 120 minutes, as indicated. The cell lysate was then collected with NP40 buffer and used for western blotting.

Induction of Myotube Atrophy via Cachectic Cytokines. For the induction of myotube atrophy via cachectic cytokines, C2C12 myotubes were incubated in serum-free media for 6 hours and then cachectic cytokines and rFibcd1 were added at the same time for a further 2 days. The cytokines used were IL-6 (recombinant mouse IL-6, R&D Systems) at 20 ng/mL (Yamaki, et al. (2012) Am. J. Physiol. Cell Physiol. 303:C135-142), TNFα (recombinant mouse TNFα, R&D Systems) at 100 ng/mL (Wang, et al. (2014) Int. Immunopharmacol. 19:206-213), and LIF (recombinant mouse LIF, R&D Systems) at 20 ng/mL (Kandarian, et al. (2018) J. Cachexia Sarcopenia Muscle 9:1109-1120). Recombinant Fibcd1 (rFibcd1) was added to serum-free media at 10 ng/mL and 100 ng/mL, as indicated.

qRT-PCR. qRT-PCR was performed as previously described (Hunt, et al. (2015) Genes Dev. 29:2475-2489). Total RNA from C2C12 myotubes was extracted by using Trizol™ (Ambion). Five hundred µg of RNA was used for reverse transcription with the iScript™ cDNA synthesis kit (Bio-Rad). qRT-PCR was done by using the iQ™ SYBR® Green supermix (Bio-Rad). Ppia was used for normalization.

Mouse Husbandry. Animals experiments were handled following animal ethics guidelines with IACUC approval. All mice were housed in a ventilated rodent-housing system with a controlled temperature (22-23° C.) and given free access to food and water.

Mice Carrying LLC (Lewis Lung Cancer) Tumors. Male C57BL/6J (The Jackson Laboratory) mice were used at 4 months of age. LLC cells (1×10⁶) were injected into the right and left flank (Hunt, et al. (2019) Cell reports 28:1268-1281 e1266; Puppa, et al. (2014) FASEB J. 28:998-1009; Talbert, et al. (2019) Cell Rep. 28:1612-1622 e1614) and tumors were allowed to grow up to ~3 weeks. Treatment with rFibcd1 began at day 16 after LLC tumor cell implantation. Specifically, mice were treated with three injections of recombinant Fibcd1 (rFibcd1) administrated intraperitoneally at 1 mg/kg of body weight or with 1% BSA in PBS on every other day for a week, at which time tumor-bearing mice were euthanized.

Melanoma Xenograft-Bearing Mice. Female 2-month-old NCI Ath/nude mice were purchased from Charles River Laboratory. MAST360B/ SJMEL030083_X2 and MAST552A/SJMEL031086_X1 cell lines were patient-derived melanoma cancer xenografts from the Childhood Solid Tumor Network collection at St. Jude Children’s Research Hospital (Stewart, et al. (2016) Dev. Biol. 411:287-293; Stewart, et al. (2017) Nature 549:96-100; Bahrami & Barnhill (2018) Pediatr. Blood Cancer 65(2); Newman, et al. (2019) Am. J. Surg. Pathol. 43:1631-1637). Cells (1.5×10⁶) of each tumor line were inoculated into the flank of nude mice. Tumors were monitored and allowed to grow for ~8 weeks. Treatment with rFibcd1 began at day 40 after implantation of MAST360B/SJMEL030083_X2 (cachectic melanoma) and MAST552A/ SJMEL031086_X1 (non-cachectic melanoma). Mice were treated with three injections of recombinant Fibcd1 (rFibcd1) or with 1% BSA in PBS administrated intraperitoneally at 3 mg/kg of body weight on every other day for a week, after which tumor-bearing mice were euthanized.

Tissue Collection. For tissue collection, mice were euthanized and the diaphragm muscle was dissected and isolated from tendons. Only the skeletal muscle portion of the dissected tissue was used for further analyses. Half of diaphragm muscle was frozen in liquid nitrogen-cooled isopentane (Sigma-Aldrich) and mounted for cryosectioning at a thickness of 10 µm whereas the other half was snap-frozen and stored at -80° C. until RNA extraction. The tumors were removed and weighed alongside.

Analysis of Myofiber Type, Size, and Number. Myofiber type analysis was done as previously described (Bloemberg & Quadrilatero (2012) PLoS ONE 7:e35273). Unfixed slides holding the sections were incubated with blocking buffer (PBS with 2% BSA and 0.1% Triton™-X100) for 1 hour before incubation with primary antibodies overnight at 4° C. The primary antibodies used were mouse IgG2b anti-myosin heavy chain type I (DSHB), mouse IgG1 anti-myosin heavy chain type IIA (DSHB), and rat anti-laminin α2 (4H8-2; Santa Cruz). The sections were then washed and incubated with secondary antibodies which were anti-mouse IgM ALEXA® 555 (Life Technologies), anti-mouse IgG1 ALEXA® 488 (Life Technologies), and anti-rat IgG ALEXA® 647 (Life Technologies). Diaphragm sections were imaged on a Nikon C2 confocal microscope with a 10x objective and the myofiber types and sizes were analyzed in an automated manner with the Nikon Elements software by using the inverse threshold of laminin α2 immunostaining to determine myofiber boundaries. The myosin heavy chain staining was used to classify type I (red), type IIA (green), and presumed type IIX/IIB myofibers (black, i.e., that were not stained for MHC I or IIA). After myofibers were classified by type, myofiber size was estimated in an automated manner by the Nikon Elements software via the Feret’s minimal diameter, a geometrical parameter for the analysis of unevenly shaped or cut objects (Bloemberg & Quadrilatero (2012) PLoS ONE 7:e35273). For the quantification of the number of myofibers, all fibers in the diaphragm cross-sections were counted based on the myofiber borders identified by laminin α2 immunostaining.

RNA-seq. Diaphragm muscles were homogenized in Trizol™ and RNA extracted by isopropanol precipitation from the aqueous phase. RNA sequencing libraries for each sample were prepared with 1 µg total RNA by using the Illumina TruSeq RNA Sample Prep v2 Kit per the manufacturer’s instructions, and sequencing was completed on the Illumina NovaSeq 6000. The 100-bp paired-end reads were trimmed, filtered against quality (Phred-like Q20 or greater) and length (50-bp or longer), and aligned to a mouse reference sequence GRCm38 (UCSC mm10) by using CLC Genomics Workbench v12.0.1 (Qiagen). For gene expression comparisons, the TPM (transcript per million) counts were obtained from the RNA-Seq Analysis tool. The differential gene expression analysis was performed via the non-parametric ANOVA using the Kruskal-Wallis and Dunn’s tests on log-transformed TPM between three replicates of experimental groups, implemented in Partek Genomics Suite v7.0 software (Partek Inc.). The average TPM counts from each experimental group for significant genes (defined as indicated in the corresponding figure legends) were further clustered in a heatmap using z-score normalization and similarity measure by correlation.

Measurement of Cytokine Levels in the Plasma of Experimental Mice. The plasma cytokine levels were measured by using the MILLIPLEX® Map mouse cytokine assay kit (Millipore) as previously done (Zhou, et al. (2011) Cell 142:531-543; Hermann, et al. (2014) World J. Methodol. 4:219-231). Blood (600 µL) was collected from the abdominal aorta of mice that carry melanoma xenografts by using 100 mL of 50 mM EDTA as an anti-coagulant and centrifuged for 20 minutes at 1,000 g. All reagents were brought to room temperature before use. Wash buffer, assay buffer, serum matrix, standard 6, quality controls 1 and 2, premixed beads, detection antibodies, and streptavidin-PE were prepared as recommended by the manufacturer. Plasma samples were diluted 1:4 by using the assay buffer. To create a homogeneous mixture, the premixed beads bottle was sonicated in water bath for 30 seconds and then vortexed for 1 minute before use. For pre-wetting, 200 µL assay buffer was pipetted into each well, the plate was covered with sealer and then shaken at 700 rpm for 10 minutes. The fluid was removed by tapping the plate on a paper towel and by centrifuging it briefly at 3,500 rpm lying top down on a paper sheet in the centrifuge. Twenty-five µL of standards 1-6 and quality controls 1 and 2 were pipetted in duplicate into the appropriate wells and 25 mL of serum matrix was added. Next, 25 mL of diluted plasma samples and 25 mL of assay buffer was pipetted into the appropriate wells. Finally, the magnetic premixed beads were vortexed for 1 minute and 25 mL was pipetted into each well. The plate was sealed, covered with aluminum foil and then shaken at 700 rpm for 16 hours at 4° C. After overnight incubation, the magnetic bead plates were washed 2 times by using assay buffer, as recommended by the manufacturer. Then, 25 mL of detection antibodies were added into each well, the plate was sealed and covered with aluminum foil and shaken at 700 rpm for 1 hour. After that, 25 mL of streptavidin-phycoerythrin was added to each well containing the 25 mL of detection antibodies. The plate was again sealed, covered with foil and incubated with agitation for 30 minutes at room temperature. Subsequently, the plate was washed 2 times again. Assay buffer (150 mL) was added to all wells and shaken for 5 minutes to resuspend the beads. Lastly, the plate was read on a LUMINEX® 200™ apparatus. The analysis was done by the LUMINEX® software using the spline curve-fitting method for calculating cytokine/chemokines concentrations in samples.

Miscellaneous Computational Analyses. Human FIBCD1 expression in muscles was examined from the following datasets: GDS2056 / 240042_at; Title: Skeletal muscle types (HG-U133B); GDS2855 / 238943_at; Title: Various muscle diseases (HG-U133B); Organism: Homo sapiens.

Statistical Analyses. Data organization, scientific graphing and statistical analyses were performed with Microsoft Excel (version 14.7.3) and GraphPad Prism (version 6). The unpaired two-tailed Student’s t-test was used to compare the means of two independent groups to each other. One-way and two-way ANOVA with post hoc testing were used for multiple comparisons of more than two groups of normally distributed data. The “n” for each experiment can be found in the figure legends and represents independently generated samples for all experiments, including myotubes and cell culture samples for in vitro assays, and individual animals for in vivo experiments. Bar graphs present the mean ±SD or ±SEM as indicated. Throughout the figures, asterisks and ampersand symbols indicate the significance of P values: *P<0.05, **P<0.01, ***P<0.001, ****P<0.0001. A significant result was defined as P<0.05.

### Example 2: RNAi and Overexpression Screening Identifies Evolutionary-Conserved Myokines that Determine Myofiber Size in Drosophila

Several myokines have been identified by transcriptomic and mass-spectrometry studies (Deshmukh, et al. (2015) J. Proteome Res. 14:4885-4895; Rai & Demontis (2016) Annu. Rev. Physiol. 78:85-107; Norheim, et al. (2011) Am. J. Physiol. Endocrinol. Metab. 301:E1013-1021) but their role in muscle wasting remains largely unexplored. Signaling pathways that regulate muscle mass in mammals play similar roles also in Drosophila, as found for insulin receptor/FoxO signaling (Piccirillo, et al. (2014) Dev. Dyn. 243:201-215; Demontis & Perrimon (2009) Development 136:983-993). On this basis, it was determined whether evolutionary-conserved myokines regulate the size of Drosophila larval body wall skeletal muscles, which each are composed of a single myofiber. As previously shown (Piccirillo, et al. (2014) Dev. Dyn. 243:201-215; Demontis & Perrimon (2009) Development 136:983-993; Hunt, et al. (2019) Cell reports 28:1268-1281 e1266), muscle-specific interventions that change the size of the body wall musculature correspondingly change the larval body size. On this basis, the larval size was used as primary screen readout to estimate the capacity of each myokine to regulate developmental muscle growth, which was followed by quantification of myofiber size. In general, the UAS/Gal4 system (Brand & Perrimon (1993) Development 118:401-415), the skeletal muscle-specific Mef2-Gal4 driver (Demontis & Perrimon (2009) Development 136:983-993; Ranganayakulu, et al. (1995) Dev. Biol. 171:169-181), and 508 UAS-RNAi transgenic fly stocks were used to knock-down 111 evolutionary-conserved myokines that have strong muscle expression (RNA-seq FPKM values ≥4), and assess the role of the same in myofiber growth.

In comparison to control RNAi lines for GFP and white, this muscle-specific RNAi screen and follow-up validation indicated that 31/508 (6.1%) RNAi lines induce myofiber atrophy similar to what is observed with overexpression of the transcription factor FoxO, indicating that these myokines are necessary to sustain the ~40-fold myofiber growth that occurs during larval development (Piccirillo, et al. (2014) Dev. Dyn. 243:201-215; Demontis & Perrimon (2009) Development 136:983-993). Conversely, similar to insulin receptor (InR) overexpression, 12/508 (2.3%) RNAi lines induced myofiber hypertrophy, indicating that these myokines normally limit developmental muscle growth, similar to mammalian myostatin (Lee (2004) Annu. Rev. Cell Dev. Biol. 20:61-86; McPherron, et al. (1997)Nature387:83-90). Further testing via immunostaining confirmed that the size of representative skeletal muscles (VL3-4) is indeed modulated by RNAi interventions that induce atrophy and hypertrophy. Importantly, transgene expression with this Mef2-Gal4 does not affect the number of syncytial nuclei (Demontis & Perrimon (2009) Development 136:983-993), indicating that the changes in myofiber size here observed do not arise from altered myoblast fusion.

Among the myokines that scored, a first class of atrophic phenotypes was induced by RNAi lines for growth factors, which indicates that they are necessary for myofiber growth in an autocrine/paracrine manner. For example, 4 different RNAi lines for the myostatin-binding protein SPARC reduced myofiber size, suggesting that SPARC normally prevents atrophy by binding to and inhibiting TGF-β ligands such as myostatin, as found in mice (Nakamura, et al. (2013) Muscle Nerve 48:791-799). Moreover, muscle-specific RNAi for the BMP2/4 homolog dpp also induced myofiber atrophy, presumably via the capacity of BMP ligands to antagonize TGF-β signaling and promote myofiber hypertrophy (Sartori, et al. (2013) Nat. Genet. 45:1309-1318).

Lastly, RNAi for the FGF homolog bnl decreased myofiber size, possibly by limiting the development of the muscle-associated trachea, which delivers oxygen to sustain muscle growth, similar to the mammalian vasculature (Hayashi & Kondo (2018) Genetics 209:367-380), whereas overexpression of the PDGF/VEGF homolog Pvf1 induced myofiber hypertrophy. Other growth factors included Wnt4 and Gpb5 (homologous to WNT9 and GPHB5, respectively), which induced hypertrophy when overexpressed. Conversely, overexpression of aos (an inhibitor of EGF receptor signaling) and myoglianin (homologous to myostatin/GDF11; Demontis, et al. (2014) Cell Reports 7:1481-1494) induced atrophy.

A second category of atrophy inducers included RNAi for the extracellular matrix proteins cg25c and mfas, respectively homologous to collagen COL4A1 and to the collagen-binding protein TGFBI. Consistently, COL4A1 mutations cause myopathy in humans (Labelle-Dumais, et al. Am. J. Human Genet. 104:847-860) whereas TGFBI loss reduces developmental myofiber growth in zebrafish (Kim & Ingham (2009) Dev. Dynamics 238:56-65). Interestingly, RNAi for lanB1, homologous to laminin LAMB2, did not significantly affect myofiber size but rather conferred an elongated shape to myofibers. Other inducers of myofiber atrophy included RNAi for the angiotensin-converting enzyme ance, for the apolipoprotein rfabg, and for CG8642, a myokine homologous to mouse Fibrinogen C Domain-Containing Protein 1 (Fibcd1). Altogether, this screen has identified evolutionary-conserved myokines that regulate myofiber size.

### Example 3: siRNAs for Mouse Fibcd1 Induce Atrophy in Mouse C2C12 Myotubes

The growth-promoting myokines identified in the Drosophila screen are evolutionary conserved. On this basis, mouse orthologs for these Drosophila myokines were selected to test whether they regulate the size of cultured mouse C2C12 myotubes. For these studies, the impact on myoblast fusion was assessed for siRNAs targeting these myokines. Subsequently, it was determined whether siRNAs transfected post-fusion induced atrophy in myotubes. Analysis at day 4 of myogenic differentiation revealed that siRNAs for Bmp1 reduced myoblast fusion whereas siRNAs targeting Wnt9a, Tgfbi, Sparc, and Fibcd1 did not.

When transfected post-fusion into C2C12 myotubes, siRNAs for Tgfbi, Sparc, and Fibcd1 induced atrophy, compared to NT control siRNAs. Importantly, via a decline in Fibcd1 mRNA levels, Fibcd1 siRNAs induced the strongest atrophy and also worsened starvation-induced atrophy. On this basis, further studies focused on Fibcd1.

### Example 4: Muscle Expression of Fibcd1 Decline in Wasting Conditions

It was subsequently determined whether Fibcd1 expression is modulated by nutrient starvation, which is a physiological condition known to induce myofiber atrophy (Graca, et al. (2013) Am. J. Physiol. 305:E1483-1494), and found that this is the case in both C2C12 myotubes and in mouse tibialis anterior muscles from mice that were nutrient starved for 24 and 48 hours. Publicly available RNA-seq datasets from the Gene Expression Omnibus were consulted to determine whether human FIBCD1 expression is modulated in the skeletal muscle of patients with disease conditions known to induce myofiber atrophy. This analysis indicated that FIBCD1 expression is lower in the skeletal muscle of old versus young persons, and that FIBCD1 mRNA levels significantly decline in the muscle of patients with juvenile dermatomyositis, amyotrophic lateral sclerosis, and Emery-Dreifuss muscular dystrophy. Taken together, these findings indicate that muscle expression of Fibcd1 declines with aging and in some other conditions associated with wasting.

### Example 5: Secreted Fibcd1 Rescues Myotube Atrophy Induced by siRNAs for Fibcd1

In Drosophila, the Fibcd1 ortholog CG8642 encodes for a secreted protein that contains a fibrinogen domain. In mice, several splice variants arise from the Fibcd1 gene. Specifically, the full-length mRNA encodes for a transmembrane version of Fibcd1 (459 amino acids, #FL, composed of exons1-7), which contains an extracellular fibrinogen domain and has been reported to work as a chitin receptor in epithelia (Moeller, et al. (2019) J. Exp. Med. 216:2689-2700; Schlosser, et al. (2009) J. Immunol. 183:3800-3809). In addition, a short Fibcd1 splice variant lacks the transmembrane region but carries the fibrinogen domain (202 amino acids, #SH, consisting of exons1,5-7) and may therefore be secreted. To demonstrate this, C-terminally FLAG-tagged short and long versions of mouse Fibcd1 were transfected into HEK293 cells and the amount of Fibcd1 recovered from the supernatant was probed with anti-FLAG antibodies. Both short (SH) and long (FL) versions of Fibcd1 were detected in similar amounts in transfected cells, compared to empty-vector (EV) controls. However, there was a ~38 kDa Fibcd1 fragment that was recovered from the culture medium of HEK293 cells transfected with full-length Fibcd1 (#FL) but not from that of cells transfected with the short version of Fibcd1 (#SH). These findings indicate that transmembrane Fibcd1 is cleaved C-terminally to generate a secreted Fibcd1 that includes the fibrinogen domain.

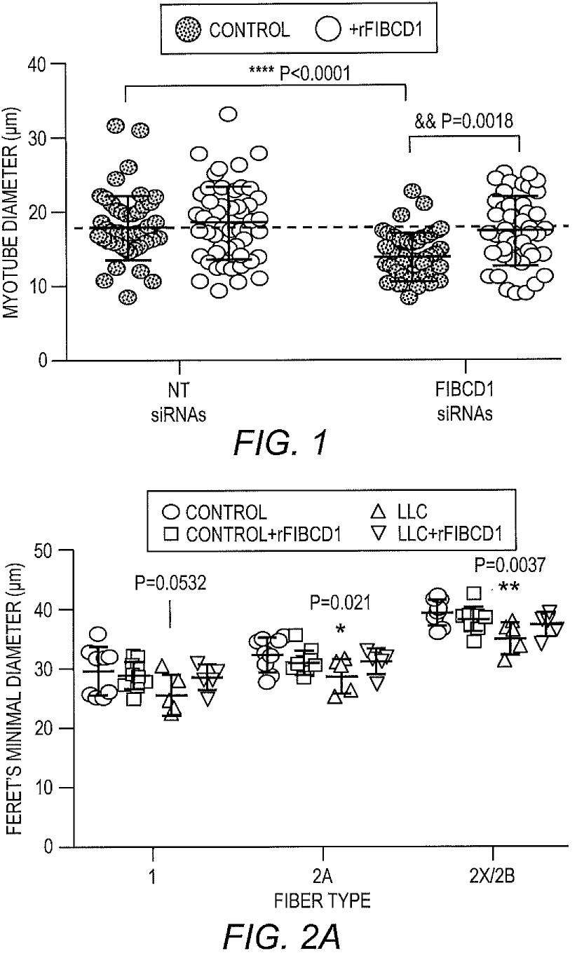

On this basis, it was next tested whether secreted Fibcd1 rescues the myotube atrophy induced by Fibcd1 siRNAs. For these studies, a ~38 kDa recombinant mouse Fibcd1 (rFibcd1; 282 C-terminal amino acids of #FL Fibcd1), similar to the extracellular fragment released by proteolytic processing of Fibcd1, was produced. Treatment with rFibcd1 rescued the myotube atrophy induced by Fibcd1 siRNAs, indicating that the myotube atrophy induced by Fibcd1 siRNAs is indeed an RNAi on-target effect, and that rFibcd1 is sufficient to reinstate myofiber size (FIG. 1).

### Example 6: rFibcd1 Rescues Myotube Atrophy Induced by Cachectic Cytokines

Cancer cells secrete inflammatory signaling factors that induce myofiber atrophy (Baracos, et al. (2018) Nat. Rev. Dis. Primers 4:17105; Fearon, et al. (2012) Cell Metab. 16:153-166; Piccirillo, et al. (2014) Dev. Dyn. 243:201-215; Bonaldo & Sandri (2013) Dis. Models Mechan. 6:25-39; Tsoli & Robertson (2013) Trends Endocrinol. Metab. 24:174-183). Because it was found that Fibcd1 is a myokine that preserves myofiber size, cachectic cytokines may induce myofiber atrophy at least in part by perturbing Fibcd1 expression.

To demonstrate this, C2C12 myotubes were treated with IL-6, LIF, and TNF-α, which are known to be upregulated in cancerous states and lead to muscle mass loss (Tsoli & Robertson (2013) Trends Endocrinol. Metab. 24:174-183). Interestingly, treatment of C2C12 myotubes with IL-6 at 20 ng/mL, LIF at 20 ng/mL, and TNF-α at 100 ng/mL led to a decrease in Fibcd1 mRNA levels, suggesting that reduced Fibcd1 expression indeed contributes to the effect of these cachectic cytokines on myofiber size.

On this basis, it was determined whether reinstating Fibcd1 levels via administration of rFibcd1 impacts myofiber atrophy induced by cachectic cytokines. This analysis indicated that in vitro treatment with IL-6, LIF, and TNF-α induced significant atrophy of C2C12 myotubes. However, treatment with rFibcd1 significantly reduced the atrophy induced by IL-6, LIF, and TNF-α. Altogether, these findings suggest that Fibcd1 mitigates wasting induced by cachectic cytokines.

### Example 7: rFibcd1 Rescues Myotube Atrophy by Promoting ERK Signaling in Muscle Cells

To examine what signaling pathways are modulated by Fibcd1 and are responsible for its effects on myotube size determination, the effect of Fibcd1 siRNAs and rFibcd1 treatments on the activity of several signaling pathways was examined with a panel of phospho antibodies. Addition of rFibcd1 to the cell culture medium of mouse C2C12 myotubes increased P-p42 and P-p44 (ERK1/2) MAPK levels after 30 minutes from stimulation, whereas no substantial changes were found in the activity of other pathways including JNK and p38 MAPK.

ERK signaling is necessary for the maintenance of skeletal muscle mass (Shi, et al. (2009) Am. J. Physiol. Cell Physiol. 296:C1040-1048) and its inhibition prevents IGF-I-induced myofiber hypertrophy (Haddad & Adams (2004) J. Appl. Physiol. 96:203-210). Moreover, ERK has been shown to be necessary to maintain adult myofiber size and their neuromuscular junctions (Benoit, et al. (2017) Nat. Med. 23:990-996; Seaberg, et al. (2015) Mol. Cell. Biol. 35:1238-1253; Murgia, et al. (2000) Nat. Cell Biol. 2:142-147). Mechanistically, ERK can preserve myofiber size via phosphorylation and inactivation of GSK3β (Argadine, et al. (2011) Am. J. Physiol. Cell Physiol. 300:C318-327; Saito, et al. (1994) Biochem. J. 303(Pt 1):27-31) and by promoting protein synthesis via activation of the MAPK-interacting kinase MNK1/2, which in turn phosphorylates the translation initiation factor eIF4E (Argadine, et al. (2011) Am. J. Physiol. Cell Physiol. 300:C318-327; Waskiewicz, et al. (1997) EMBO J. 16:1909-1920; Waskiewicz, et al. (1999) Mol. Cell. Biol. 19:1871-1880).

On the basis of these studies, Fibcd1 may modulate myofiber size at least in part via ERK signaling. To demonstrate this, C2C12 myotubes were treated with Fibcd1 siRNAs, rFibcd1, and/or Pyrazolylpyrrole, a pharmacological inhibitor of ERK (Hunt, et al. (2015) Genes Dev. 29:2475-2489; Aronov, et al. (2007) J. Med. Chem. 50:1280-1287; Junttila, et al. (2008) FASEB J. 22:954-965). By using low doses of Pyrazolylpyrrole (2.5 ng/mL), it was observed that Pyr has no major impact on myotube size in normal conditions. However, Pyrazolylpyrrole prevents rFibcd1 from rescuing myotube atrophy induced by Fibcd1 siRNAs, indicating that rFibcd1 reinstates myotube size via its capacity to increase ERK activity.

### Example 8: rFibcd1 Does Not Promote ERK Signaling in a Set of Cancer Cells

ERK is a major driver of tumorigenesis (Dhillon, et al. (2007) Oncogene 26:3279-3290) and therefore interventions that combat muscle wasting via ERK signaling may be hindered by the side effect of promoting cancer cell proliferation (Musaro, et al. (2001) Nat. Genet. 27:195-200). On this basis, it was determined whether rFibcd1 increases P-ERK levels also in cancer cells. For this purpose, P-ERK levels were examined in Lewis lung carcinoma (LLC) (Kaplan, et al. (2005) Nature 438:820-827), 4T1 breast cancer (Labelle, et al. (2014) Proc. Natl. Acad. Sci. USA 111:E3053-3061; Jia, et al. (2019) EMBO J. 38:e101302), and Saos-2 osteosarcoma cells (Yu, et al. (2017) BMC Cancer 17:78). Different from C2C12 myotubes, western blot analyses indicated that rFibcd1 does not promote ERK signaling in these cancer cells. Altogether these findings indicate that rFibcd1 combats myofiber atrophy by inducing ERK signaling in muscle but not in cancer cells. Accordingly, rFibcd1 provides an effective therapy for treating cachexia-induced myofiber atrophy without fueling cancer growth.

### Example 9: rFibcd1 Rescues LLC Cancer-Induced Myofiber Atrophy in the Diaphragm Muscle of Mice

To test the therapeutic potential of rFibcd1 in vivo, an established model of cancer cachexia was employed wherein the LLC cells are subcutaneously injected (Puppa, et al. (2014) FASEB J. 28:998-1009; Talbert, et al. (2019) Cell Rep. 28:1612-1622 e1614; Hunt, et al. (2019) Cell Reports 28:1268-1281 e1266).

For these experiments, ~10⁶ LLC cells were injected subcutaneously in each flank and allowed to grow for ~3 weeks. One week before reaching the endpoint, tumor-bearing mice were randomly allocated to receive 3 intraperitoneal injections of rFibcd1 (every other day; 1 mg/kg of rFibcd1 in PBS with 1% BSA) or mock (PBS with 1% BSA). Cohorts of isogenic C57BL/6J control mice devoid of tumors were similarly injected with either rFibcd1 or PBS.

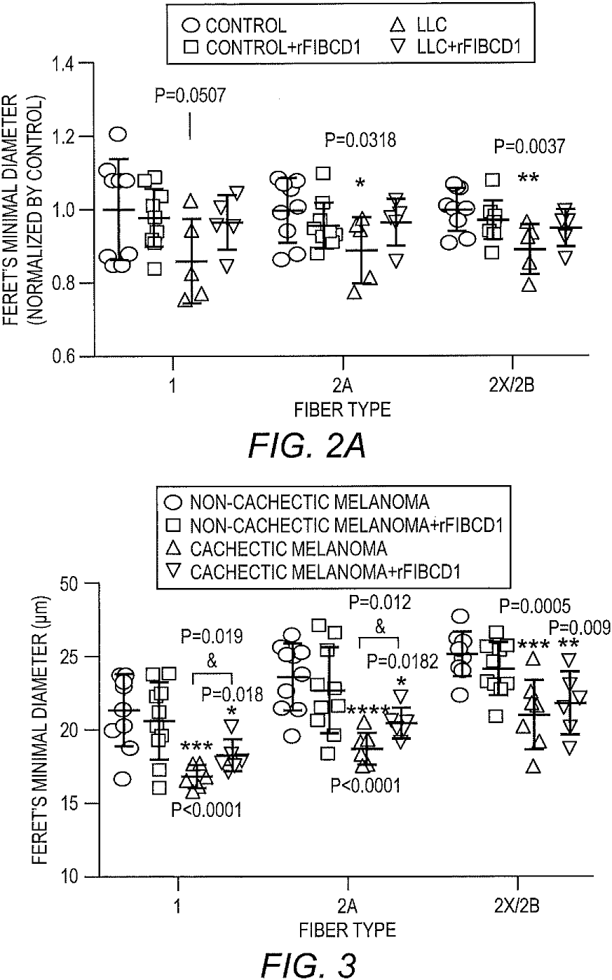

Because the diaphragm muscle is key for respiration, wasting of the diaphragm is a key determinant of cachexia-associated mortality (Roberts, et al. (2013) FASEB J. 27:2600-2610; Smith, et al. (2016) Am. J. Physiol. Regul. Integr. Comp. Physiol. 310:R707-710). Therefore, interventions that preserve the size of diaphragm myofibers are key for reducing mortality associated with cachexia and other critical illnesses (Berger, et al. (2016) J. Cachexia Sarcopenia Muscle 7:403-412). Thus, it was determined whether rFibcd1 rescues cancer-induced wasting of diaphragm myofibers and it was observed that this is the case (FIGS. 2A-2B). Specifically, LLC cancer cells induce atrophy of all myofiber types (1, 2a, and 2x/2b) but this is rescued by intraperitoneal rFibcd1 injection (FIGS. 2A-2B), as indicated by the Feret’s minimal diameter, a geometrical parameter indicative of myofiber size (Briguet, et al. (2004) Neuromuscul. Disord. 14:675-682). However, rFibcd1 injection does not affect myofiber size in wild-type (non-cachectic) conditions (FIGS. 2A-2B), indicating that Fibcd1 maintains steady-state myofiber size but does not induce hypertrophy.

Because myofiber types have different defining sizes, the reduction in myofiber atrophy due to rFibcd1 may be explained via its capacity to promote a shift from type 1 to type 2a or 2x/2b myofibers, which are bigger (Schiaffino & Reggiani (2011) Physiol. Rev. 91:1447-1531). However, immunostaining of diaphragm muscles with antibodies for distinct myosin heavy chain isoforms revealed that there are no changes in the relative abundance of different myofiber types present in the diaphragm of mice treated with rFibcd1 versus controls.

The distribution of myofiber sizes present in the diaphragm was subsequently determined. For each myofiber type, LLC cancer cells induced a shift toward smaller sizes, which was rescued by rFibcd1.

The capacity of rFibcd1 to resolve myofiber atrophy in cancer-bearing mice may arise via its capacity to contrast cachectic changes in muscle or, alternatively, via its direct action on cancer cells. Therefore, the body weight and tumor-free body mass of mice was determined and it was observed that they declined in mice injected with LLC cancer cells. However, body weight and tumor-free body mass did not differ in response to intraperitoneal rFibcd1 injection. Consistently, LLC tumor masses were similar in mice treated with rFibcd1 versus controls.

Altogether, these findings indicate that rFibcd1 resolves myofiber atrophy induced by cancer cachexia and that this is not due to an effect of rFibcd1 on cancer growth.

### Example 10: rFibcd1 Rescues Muscle Transcriptional Changes Induced by LLC Cancers

It was subsequently determined whether rFibcd1 rescues the molecular changes associated with cachexia-induced myofiber atrophy in the diaphragm. In parallel with the preservation of myofiber size, RNA-sequencing revealed that LLC-induced cachexia is characterized by an increase in the expression of many genes whereas relatively few have reduced expression. However, rFibcd1 significantly mitigates LLC-induced transcriptional changes in the diaphragm muscle, compared to mock injections.

Further analysis of 247 significantly regulated genes in LLC versus LLC+rFibcd1 (p<0.05 and -0.5>log2R>0.5) revealed that several categories of genes involved in immunity are overrepresented among LLC-upregulated genes, including the chemokine Cxcl13 and receptors for inflammatory cachectic cytokines. Together, these findings indicate that cachectic factors produced by LLC cells, such as IL-6, LIF, and TNF-α (Puppa, et al. (2014) FASEB J. 28:998-1009; Zhang, et al. (2017) Sci. Rep. 7:2273), induce inflammatory signaling in the diaphragm muscle and that intraperitoneal injection of rFibcd1 can largely impede such transcriptional changes.

Previous studies have shown that muscle proteolysis is a key mechanism responsible for myofiber atrophy induced by cancer (Piccirillo, et al. (2014) Dev. Dyn. 243:201-215; Bonaldo & Sandri (2013) Dis. Models Mechan. 6:25-39). In this respect, further analysis revealed that LLC cancer cells increase the expression of cathepsins in the diaphragm muscle, such as Ctss, Ctse, Ctsw, Ctsz, Ctsc, Ctsh, and Ctso. Because cathepsin are lysosomal proteolytic enzymes that have been previously implicated in myofiber atrophy (Lecker, et al. (2004) FASEB J. 18:39-51; Ruff & Secrist (1984) J. Clin. Invest. 73:1483-1486; Tjondrokoesoemo, et al. (2016) J. Biol. Chem. 291:9920-9928; Zhao, et al. (2007) Cell Metab. 6:472-483), they likely contribute to the proteolysis responsible for LLC cancer-induced muscle mass loss. However, rFibcd1 largely prevented the upregulation of cathepsin expression by LLC cancers. Other proteolytic enzymes were also similarly regulated by LLC cancers and rFibcd1, such as Napsa, a member of the peptidase A1 family of aspartic proteases. Moreover, ubiquitin D (Ubd) expression was induced by LLC cancers but repressed by rFibcd1 and may sustain proteolysis via the ubiquitin-proteasome system. Lastly, ubiquitin ligases were also regulated and are known to have important roles in muscle proteolysis during wasting (Bonaldo & Sandri (2013) Dis. Models Mechan. 6:25-39; Lecker, et al. (2004) FASEB J. 18:39-51; Gomes et al. (2001) Proc. Natl. Acad. Sci. USA 98:14440-14445; Bodine, et al. (2014) Am. J. Physiol. Endocrinol. Metab. 307:E469-484). Interestingly, some of them were upregulated in the diaphragm of mice with LLC cancers but this was prevented by rFibcd1 injection. These included Hgs and Syvnl, which were previously shown to negatively regulate myofiber size (Hunt, et al. (2019) Cell Reports 28:1268-1281 e1266). However, LLC cancers did not significantly induce other ubiquitin ligases involved in myofiber atrophy (Bonaldo & Sandri (2013) Dis. Models Mechan. 6:25-39; Lecker, et al. (2004) FASEB J. 18:39-51; Gomes et al. (2001) Proc. Natl. Acad. Sci. USA 98:14440-14445; Bodine, et al. (2014) Am. J. Physiol. Endocrinol. Metab. 307:E469-484), such as Fbxo32/Atrogin-1 and Trim63/MuRF1. Although relatively few genes were downregulated in response to LLC cancers, there were some that have been previously associated with myofiber hypertrophy, such as Pparg1a/PGC1a (Ruas, et al. (2012) Cell 151:1319-1331), which was downregulation in LLC but not in LLC+rFibcd1 versus control.

Altogether, these findings indicate that rFibcd1 hinders some of the molecular changes associated with cancer cachexia in the diaphragm, including expression of genes already implicated in myofiber atrophy via their roles in inflammation, proteolysis, and other processes.

### Example 11: rFibcd1 Rescues Myofiber Atrophy Induced by a Patient-Derived Melanoma Xenograft

LLC cancer-induced cachexia is an established disease model that has led to many insights into the mechanisms of muscle wasting in cancer (Hunt, et al. (2019) Cell Reports 28:1268-1281 e1266; Puppa, et al. (2014) FASEB J. 28:998-1009; Talbert, et al. (2019) Cell Rep. 28:1612-1622 e1614). However, there is growing appreciation that patient-derived cancer xenografts provide cachexia models that more closely mimic disease progression in humans (Talbert, et al. (2019) Cell Rep. 28:1612-1622 e1614; Delitto, et al. (2017) Oncotarget 8:1177-1189). On this basis, the capacity of rFibcd1 to mitigate myofiber atrophy induced by a patient-derived xenograft was examined. For this purpose, an orthotopic patient-derived spitzoid melanoma xenograft was identified from the Childhood Solid Tumor Network collection established at St. Jude Children’s Research Hospital (Stewart, et al. (2016) Dev. Biol. 411:287-293; Stewart, et al. (2017) Nature 549:96-100; Bahrami & Barnhill (2018) Pediatr. Blood Cancer 65; Newman, et al. (2019) Nat. Med. 25:597-602; Newman, et al. (2019) Am. J. Surg. Pathol. 43:1631-1637), which induces severe (~25%) muscle mass loss.

On this basis, it was determined whether rFibcd1 impedes myofiber atrophy induced by a pediatric spitzoid melanoma xenograft (MAST360B/SJMEL030083_X2), compared to a control melanoma that does not induce muscle wasting (MAST552A/SJMEL031086_X1). Compared to the commonly-used LLC model of cancer-induced cachexia (FIGS. 2A-2B), the spitzoid melanoma model (MAST360B) here used induces severe myofiber atrophy of several muscles, including the diaphragm (~25% versus ~10% loss).

For these experiments, ~10⁶ pediatric melanoma xenograft cells were injected subcutaneously in the flank and allowed to grow for ~8 weeks. To test the efficacy of rFibcd1, 3 intraperitoneal injections (every other day, with 3 mg/kg of rFibcd1 in PBS with 1% BSA) were done in the last week of xenograft growth before euthanasia in half of the mice carrying xenografts whether the other half received mock injections (PBS with 1% BSA).

By examining the size of myofibers in the diaphragm muscle of these mice, it was observed that the cachectic melanoma induces significant atrophy, as indicated by the average Feret’s minimal diameter, compared to mice injected with a control non-cachectic melanoma. Importantly, all myofiber types (1, 2a, 2x/2b) were affected, indicating the cachectic factors released by this spitzoid melanoma have the general capacity to induce wasting (FIG. 3). However, rFibcd1 injection significantly reduced the atrophy induced by cancer cachexia in type 1 and 2a myofibers, whereas smaller effects were seen for type 2b myofibers (FIG. 3). Similar results were found by examining the average Feret’s minimal diameter values normalized to control. These changes in myofiber size were not due to a variation in the percentage of myofiber types present in the diaphragm.

In light of these results, the relative frequencies of myofibers in the diaphragm muscle were assessed. Analysis of type 1, type 2a, and type 2x/2b revealed that the cachectic melanoma shifts the distribution of myofiber sizes towards lower values compared to a control non-cachectic melanoma. However, rFibcd1 partially reinstated and/or prevented this shift in myofiber sizes, in particular for type 2a myofibers. Considering that type 2a myofibers are the most abundant in the diaphragm muscle (~60% of all myofibers) and are also abundant (-50%) in the diaphragm in humans (Lieberman, et al. (1973) J. Appl. Physiol. 34:233-237; Polla, et al. (2004) Thorax 59:808-817), these findings suggest that rFibcd1 may provide a significant means for preserving myofiber size in the diaphragm of cancer patients.

Further analyses revealed that the anti-cachectic effects of intraperitoneal injections of rFibcd1 on the diaphragm muscle were not due to a systemic decrease in cancer growth and body wasting but were rather due to local effects on the diaphragm muscle. Specifically, body weight significantly decreased in mice that carried the MAST360B/SJMEL030083_X2 cachectic melanoma compared to mice injected with the MAST552A/SJMEL031086_X1 non-cachectic melanoma. However, body weight equally declined in mice that carried the MAST360B/SJMEL030083_X2 cachectic melanoma and that received either rFibcd1 or mock intraperitoneal injections. The same conclusions were reached also via the analysis of the tumor-free body mass. Moreover, intraperitoneal rFibcd1 injection did not impact tumor growth. Altogether, these findings indicate that reduction in cancer-induced atrophy of diaphragm myofibers is not due to systemic effects of intraperitoneal injection of rFibcd1 on body wasting and tumor growth. Lastly, circulating inflammatory cytokines were inconsistently modulated by rFibcd1, i.e., whether the levels of some increased (e.g., IL5) other decreased (e.g., IL6) in response to rFibcd1, suggesting that the capacity of rFibcd1 to decrease cancer-induced myofiber atrophy primarily results from its action on muscle cells.

### Example 12: rFibcd1 Rescues Muscle Transcriptional Changes Induced by Cachectic Melanomas

Because rFibcd1 rescues myofiber atrophy induced by patient-derived xenografts of cachectic melanomas, it was determined whether rFibcd1 rescues the muscle transcriptional changes associated with myofiber atrophy. RNA-sequencing revealed that rFibcd1 regulates the expression of several genes, some of which are modulated also in cachectic versus non-cachectic melanomas. Altogether, as observed for transcriptional changes due to LLC cancers, treatment with rFibcd1 reduces the magnitude of gene expression changes induced in the diaphragm muscle in response to cachectic versus non-cachectic melanomas.

To further examine the impact of rFibcd1 on muscle cachectic changes, the GO term categories were examined that are enriched in genes that are significantly regulated in the muscle of mice with cachectic melanomas but not in the muscle of mice with cachectic melanomas upon rFibcd1 treatment. Examples of genes differentially modulated upon rFibcd1 treatment include the myokine Apelin (Apln) and its receptor (Aplnr), which are induced by rFibcd1 and have been previously implicated in preserving muscle mass in response to exercise (Vinel, et al. (2018) Nat. Med. 24:1360-1371). Conversely, the ubiquitin ligases Mylip and Fbxo31, which may promote muscle proteolysis associated with cachexia (Milan, et al. (2015) Nat. Commun. 6:6670), were repressed by rFibcd1,similar to other genes (Ace and Arrdc2) previously implicated in atrophy (Onder, et al. (2006) Curr. Pharm. Des. 12:2057-2064; Gordon, et al. (2019) Physiol. Genomics 51:208-217). Together, these findings indicate that intraperitoneal injection of rFibcd1 can impede some of the transcriptional changes associated with melanoma-induced cachexia.

### Example 13: Drug-Induced Muscle Atrophy

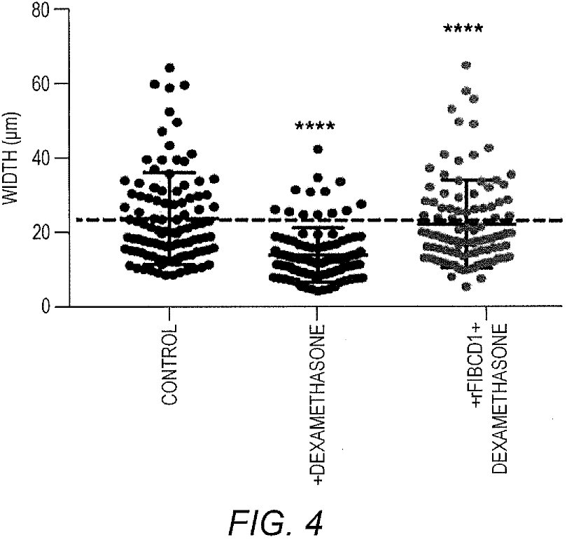

Glucocorticoids, such as dexamethasone (DEX) are potent immunosuppressants and anti-inflammatory agents, and are widely used to treat various clinical conditions, including asthma and autoimmune diseases. However, glucocorticoid-induced myopathy is a serious side effect, and indeed is the most common type of drug-induced myopathy. Accordingly, muscle cells were treated with dexamethasone, with or with rFibcd1,to assess the effect on rFibcd1 on drug-induced myofiber atrophy. This analysis indicated that rFibcd1 treatment could rescue dexamethasone-induced myofiber atrophy (FIG. 4).

What is claimed is:

1. A recombinant Fibrinogen C Domain Containing 1 (Fibcd1) fragment, variant and/or derivative thereof, wherein said fragment comprises a fibrinogen-related domain.

2. The recombinant Fibcd1 fragment of claim 1, wherein said fragment is less than 400 amino acid residues in length.

3. The recombinant Fibcd1 fragment of claim 1, wherein said fragment comprises residues 241-457 of SEQ ID NO:2, or an ortholog thereof.

4. A vector comprising a nucleic acid sequence encoding the recombinant Fibcd1 fragment of claim 1.

5. The vector of claim 4, further comprising a nucleotide sequence encoding a muscle-specific promoter, enhancer, or both.

6. A host cell comprising the vector of claim 4.

7. A pharmaceutical composition comprising the recombinant Fibcd1 fragment of claim 1 in admixture with a pharmaceutically acceptable carrier.

8. A fusion protein comprising a Fibrinogen C Domain Containing 1 (Fibcd1) protein and a second polypeptide.

9. The fusion protein of claim 8, wherein said Fibcd1 protein is a Fibcd1 fragment, Fibcd1 variant and/or Fibcd1 derivative.

10. The fusion protein of claim 8, wherein the second polypeptide is an epitope or cell-penetrating peptide.

11. A pharmaceutical composition comprising the fusion protein of claim 8 in admixture with a pharmaceutically acceptable carrier.

12. A modified RNA molecule comprising a nucleic acid sequence encoding the recombinant fragment of claim 1.

13. A method of treating muscle atrophy in a subject comprising administering to a subject in need of treatment an effective amount of the recombinant Fibcd1 fragment, variant and/or derivative of claim 1 thereby treating the subject’s muscle atrophy.

14. The method of claim 13, wherein the muscle atrophy is associated with aging, injury, disuse, cachexia, nutritional or metabolic derangements, vascular insufficiency, drug treatment or a neuromuscular disorder or disease.

15. A method of treating muscle atrophy in a subject comprising administering to a subject in need of treatment an effective amount of the vector of claim 4 thereby treating the subject’s muscle atrophy.

16. A method of treating muscle atrophy in a subject comprising administering to a subject in need of treatment an effective amount of the fusion protein of claim 8 thereby treating the subject’s muscle atrophy.

17. A method of treating muscle atrophy in a subject comprising administering to a subject in need of treatment an effective amount of the modified RNA of claim 12 thereby treating the subject’s muscle atrophy.


SEQUENCE LISTING (21 sequences)

SEQ ID NO: 1 - DNA, 1386 bp, Artificial Sequence
  Synthetic peptide
atggtcaacgaccggtggaagaccatgggcggcgctgcccaacttgaggaccggccgcgcgacaagccgcagcggccgagctgcggctacgtgctgtgcaccgtgctgctggccctggctgtgctgctggctgtagctgtcaccggtgccgtgctcttcctgaaccacgcccacgcgccgggcacggcgcccccacctgtcgtcagcactggggctgccagcgccaacagcgccctggtcactgtggaaagggcggacagctcgcacctcagcatcctcattgacccgcgctgccccgacctcaccgacagcttcgcacgcctggagagcgcccaggcctcggtgctgcaggcgctgacagagcaccaggcccagccacggctggtgggcgaccaggagcaggagctgctggacacgctggccgaccagctgccccggctgctggcccgagcctcagagctgcagacggagtgcatggggctgcggaaggggcatggcacgctgggccagggcctcagcgccctgcagagtgagcagggccgcctcatccagcttctctctgagagccagggccacatggctcacctggtgaactccgtcagcgacatcctggatgccctgcagagggaccgggggctgggccggccccgcaacaaggccgaccttcagagagcgcctgcccggggaacccggccccggggctgtgccactggctcccggccccgagactgtctggacgtcctcctaagcggacagcaggacgatggcgtctactctgtctttcccacccactacccggccggcttccaggtgtactgtgacatgcgcacggacggcggcggctggacggtgtttcagcgccgggaggacggctccgtgaacttcttccggggctgggatgcgtaccgagacggctttggcaggctcaccggggagcactggctagggctcaagaggatccacgccctgaccacacaggctgcctacgagctgcacgtggacctggaggactttgagaatggcacggcctatgcccgctacgggagcttcggcgtgggcttgttctccgtggaccctgaggaagacgggtacccgctcaccgtggctgactattccggcactgcaggcgactccctcctgaagcacagcggcatgaggttcaccaccaaggaccgtgacagcgaccattcagagaacaactgtgccgccttctaccgcggtgcctggtggtaccgcaactgccacacgtccaacctcaatgggcagtacctgcgcggtgcgcacgcctcctatgccgacggcgtggagtggtcctcctggaccggctggcagtactcactcaagttctctgagatgaagatccggccggtccgggaggaccgctag

SEQ ID NO: 2 - protein, 461 aa, Artificial Sequence
  Synthetic peptide
MVNDRWKTMGGAAQLEDRPRDKPQRPSCGYVLCTVLLALAVLLAVAVTGAVLFLNHAHAPGTAPPPVVSTGAASANSALVTVERADSSHLSILIDPRCPDLTDSFARLESAQASVLQALTEHQAQPRLVGDQEQELLDTLADQLPRLLARASELQTECMGLRKGHGTLGQGLSALQSEQGRLIQLLSESQGHMAHLVNSVSDILDALQRDRGLGRPRNKADLQRAPARGTRPRGCATGSRPRDCLDVLLSGQQDDGVYSVFPTHYPAGFQVYCDMRTDGGGWTVFQRREDGSVNFFRGWDAYRDGFGRLTGEHWLGLKRIHALTTQAAYELHVDLEDFENGTAYARYGSFGVGLFSVDPEEDGYPLTVADYSGTAGDSLLKHSGMRFTTKDRDSDHSENNCAAFYRGAWWYRNCHTSNLNGQYLRGAHASYADGVEWSSWTGWQYSLKFSEMKIRPVREDR

SEQ ID NO: 3 - protein, 217 aa, Artificial Sequence
  Synthetic peptide
PRDCLDVLLSGQQDDGVYSVFPTHYPAGFQVYCDMRTDGGGWTVFQRREDGSVNFFRGWDAYRDGFGRLTGEHWLGLKRIHALTTQAAYELHVDLEDFENGTAYARYGSFGVGLFSVDPEEDGYPLTVADYSGTAGDSLLKHSGMRFTTKDRDSDHSENNCAAFYRGAWWYRNCHTSNLNGQYLRGAHASYADGVEWSSWTGWQYSLKFSEMKIRPV

SEQ ID NO: 4 - protein, 15 aa, Artificial Sequence
  Synthetic peptide
RQIKWFQNRRMKWKK

SEQ ID NO: 5 - protein, 11 aa, Artificial Sequence
  Synthetic peptide
YGRKKRRQRRR

SEQ ID NO: 6 - protein, 11 aa, Artificial Sequence
  Synthetic peptide
YARAAARQARA

SEQ ID NO: 7 - protein, 13 aa, Artificial Sequence
  Synthetic peptide
GRRRRRRRRRPPQ

SEQ ID NO: 8 - protein, 10 aa, Artificial Sequence
  Synthetic peptide
RRRRRRRRRR

SEQ ID NO: 9 - protein, 18 aa, Artificial Sequence
  Synthetic peptide
RGGRLSYSRRRFSTSTGR

SEQ ID NO: 10 - protein, 10 aa, Artificial Sequence
  Synthetic peptide
RRLSYSRRRF

SEQ ID NO: 11 - protein, 12 aa, Artificial Sequence
  Synthetic peptide
PIRRRKKLRRLK

SEQ ID NO: 12 - protein, 12 aa, Artificial Sequence
  Synthetic peptide
RRQRRTSKLMKR

SEQ ID NO: 13 - protein, 15 aa, Artificial Sequence
  Synthetic peptide
RRRRNRTRRNRRRVR

SEQ ID NO: 14 - protein, 19 aa, Artificial Sequence
  Synthetic peptide
KMTRAQRRAAARRNRWTAR

SEQ ID NO: 15 - protein, 13 aa, Artificial Sequence
  Synthetic peptide
TRRQRTRRARRNR

SEQ ID NO: 16 - protein, 13 aa, Artificial Sequence
  Synthetic peptide
GRKKRRQRRRPPQ

SEQ ID NO: 17 - protein, 27 aa, Artificial Sequence
  Synthetic peptide
GWTLNSAGYLLGKINLKALAALAKKIL

SEQ ID NO: 18 - protein, 17 aa, Artificial Sequence
  Synthetic peptide
KLALKLALKLALALKLA

SEQ ID NO: 19 - protein, 27 aa, Artificial Sequence
  Synthetic peptide
MGLGLHLLVLAAALQGAWSQPKKKRKV

SEQ ID NO: 20 - protein, 27 aa, Artificial Sequence
  Synthetic peptide
GALFLGWLGAAGSTMGAWSQPKKKRKV

SEQ ID NO: 21 - protein, 21 aa, Artificial Sequence
  Synthetic peptide
FLGAAGSTMGAWSQPKKKRKV